(19) United States
(12) Patent Application Publication
(10) Pub. No.: US 2024/0407954 A1
(43) Pub. Date: Dec. 12, 2024

(54) NEGATIVE PRESSURE WOUND THERAPY SYSTEM

(71) Applicant: KCI Manufacturing Unlimited Company, Athlone, Co., Westmeath (IE)

(72) Inventors: Benjamin A. PRATT, Bracknell (GB); Robert HOWARD, Bracknell (GB); Thomas A. EDWARDS, Bracknell (GB)

(21) Appl. No.: 18/691,395

(22) PCT Filed: Aug. 23, 2022
(86) PCT No.: PCT/IB2022/057871

§ 371 Date: Mar. 12, 2024

Related U.S. Application Data
(60) Provisional application No. 63/244,448, filed on Sep. 15, 2021.

(51) Int. Cl. A61F 13/0203; A61M 1/00

(52) U.S. Cl. CPC A61F 13/0203; A61M 1/80; A61M 2205/3368

(57) ABSTRACT

Apparatuses, systems, and methods for providing negative pressure therapy are described. The system includes a dressing configured to be positioned adjacent to a tissue site, and a therapy unit. The therapy unit includes a pump module and a forced air module. The pump module includes a piezoelectric pump sealed between a pump casing, and a lid. The forced air module includes a forced air device configured to generate a fluid flow and a pathway enclosure configured to form a fluid path across the lid. The system further includes a canister with a pathway connection and an airflow pathway extending through the canister. The airflow pathway is lined with an evaporative membrane configured to allow evaporated fluids in the canister to escape to ambient air. The forced air module directs the fluid flow into the canister through the pathway connection and over the evaporative membrane to maximize fluid evaporation.

### CROSS-REFERENCE TO RELATED APPLICATIONS

This application is a U.S. National Stage Entry of International Application No. PCT/IB2022/057871, filed Aug. 23, 2022, which claims the benefit of priority to U.S. Provisional Application No. 63/244,448, filed on Sep. 15, 2021, which is incorporated herein by reference in its entirety.

### TECHNICAL FIELD

The invention set forth in the appended claims relates generally to tissue treatment systems and more particularly, but without limitation, to pump and canister efficiency.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for increasing pump efficiency and total fluid handling in a negative-pressure therapy environment are set forth in the appended claims. Illustrative embodiments are also provided to enable a person skilled in the art to make and use the claimed subject matter.

For example, in some embodiments, a therapy system is described. The therapy system can include a dressing configured to be positioned adjacent to a tissue site, and a therapy unit. The therapy unit can include a pump module and a forced air module. The pump module can include a piezoelectric pump sealed between a pump casing, and a lid. The forced air module can include a forced air device configured to generate a fluid flow and a pathway enclosure configured to form a fluid path across the lid. The system can further include a canister with a pathway connection and an airflow pathway extending through the canister. The airflow pathway can be lined with an evaporative membrane configured to allow evaporated fluids in the canister to escape to ambient air.

The lid can comprise a plurality of ribs extending opposite the pump casing. The plurality of ribs can extend from the third wall to the fourth wall such that the ribs are parallel to the first wall and the second wall of the pump casing. The forced air device can be configured to direct the fluid flow through the ribs of the lid. In some embodiments, the forced air device can be positioned relative to the pump casing at a non-perpendicular angle. In these embodiments, the fluid flow can define a curved pathway.

Other example embodiments describe a pump module for use in a negative-pressure system. The pump module can include a pump casing, a piezoelectric pump, a lid, and a temperature sensor. The pump casing can include a base with a first side and a second side opposite the first side. A first wall can protrude from the first side of the base, a second wall can be opposite the first wall, a third wall can connect the first wall and the second wall, and a fourth wall can be opposite the third wall. A walled enclosure can be disposed between the four walls and can define a pump seat that can house the piezoelectric pump. There can be a first bore depending through the base from the pump seat on the first side of the base to the second side of the base. The first bore can be connected to a first conduit that has at least one lumen fluidly coupled to the first bore. There can be a second bore depending through the base that can be connected to a second conduit that has at least one lumen fluidly coupled to the second bore. The piezoelectric pump can be coupled to the first side of the base at the pump seat. The lid can be coupled to the pump casing and can be configured to seal the piezoelectric pump between the pump casing and the lid. The temperature sensor can be disposed between the pump casing and the lid. In some embodiments, the temperature sensor can be coupled to the piezoelectric pump opposite the pump seat. The pump casing and the lid can be configured to optimize the efficiency of the piezoelectric pump.

The pump casing can include an opening that extends through the first wall. The piezoelectric pump can include a projection with an electrical connection that can be configured to extend through the opening. The electrical connection can allow the pump module to be coupled to potential sources outside of the pump casing.

The pump casing can comprise an insulating material such as thermoplastic, foam, or a vacuum wall. The lid can be a heat sink and can comprise a plurality of ribs extending away from the pump module opposite the pump casing. The ribs can extend from the third wall to the fourth wall such that they are parallel to the first wall and the second wall of the pump casing. The lid can comprise a thermally conductive material such as aluminum or copper.

In other embodiments, a system for negative-pressure therapy can include a dressing configured to be positioned adjacent to a tissue site, and a therapy unit. The therapy unit can include a pump module. The pump module can include a temperature sensor and a piezoelectric pump sealed between a pump casing, and a lid. The pump casing can include a base with a first side and a second side opposite the first side. A first wall can protrude from the first side of the base, a second wall can be opposite the first wall, a third wall can connect the first wall and the second wall, and a fourth wall can be opposite the third wall. A walled enclosure can be disposed between the four walls and define a pump seat that houses the piezoelectric pump. There can be a first bore depending through the base from the pump seat on the first side of the base to the second side of the base. The first bore can be connected to a first conduit that has at least one lumen fluidly coupled to the first bore. There can be a second bore depending through the base that can be connected to a second conduit that has at least one lumen fluidly coupled to the second bore. The piezoelectric pump can be coupled to the first side of the base at the pump seat. The lid can be coupled to the pump casing and can be configured to seal the piezoelectric pump between the pump casing and the lid. The temperature sensor can be disposed between the pump casing and the lid. In some embodiments, the temperature sensor can be coupled to the piezoelectric pump opposite the pump scat.

The therapy unit can further include a forced air device positioned proximate to the pump module and a pathway enclosure configured to enclose the forced air device and the lid. The pathway enclosure can allow the forced air device to direct a fluid flow across the lid. The therapy unit can also include a fluid storage canister configured to be coupled to a wall of the therapy unit and configured to be in fluid communication with the pump module.

A method for generating negative pressure is also described herein. Some example embodiments include a dressing that can be positioned adjacent to a tissue site and a therapy unit that can be coupled to the dressing. The therapy unit can include a pump module with a pump casing, a piezoelectric pump, a lid, and a temperature sensor. The pump casing can include a base with a first side and a second side opposite the first side. A first wall can protrude from the first side of the base, a second wall can be opposite the first wall, a third wall can be connected the first wall and the second wall, and a fourth wall can be opposite the third wall. A walled enclosure can be disposed between the four walls and can define a pump seat which can house the piezoelectric pump. There can be a first bore depending through the base from the pump seat on the first side of the base to the second side of the base. The first bore can be connected to an exhaust conduit that can have at least one lumen fluidly coupled to the first bore. There can be a second bore depending through the base that can be connected to an intake conduit that has at least one lumen fluidly coupled to the second bore. The piezoelectric pump can be coupled to the first side of the base at the pump seat. The lid can be coupled to the pump casing and can be configured to seal the piezoelectric pump between the pump casing and the lid. The temperature sensor can be disposed between the pump casing and the lid. The therapy unit can further include a diaphragm pump positioned proximate to the pump module. The diaphragm pump can be configured to be in fluid communication with the tissue site.

The piezoelectric pump and the diaphragm pump can be started so that the tissue site can be drawn to a desired negative-pressure. The intake conduit can be used to draw fluid from the tissue site into the pump module. The exhaust conduit can be used to allow fluid in the pump module to escape the pump module.

The therapy unit can further include a forced air device that can be positioned proximate to the pump module. The forced air device can be turned on to regulate an internal temperature of the pump module. The temperature sensor can monitor the internal temperature of the pump module. If the internal temperature of the pump module is greater than a predetermined temperature, the forced air device can be started. The forced air device can direct a fluid flow over the lid where the fluid flow can be configured to reduce the internal temperature of the pump module. If the internal temperature is less than a predetermined temperature, the forced air device can be stopped.

A fluid storage method is also described herein. Some example embodiments include a dressing that can be positioned adjacent to a tissue site and a therapy unit that can be coupled to the dressing. The therapy unit can include a pump module with a pump casing, a piezoelectric pump, a lid, and a temperature sensor. The pump casing can include a base with a first side and a second side opposite the first side. A first wall can protrude from the first side of the base, a second wall can be opposite the first wall, a third wall can be connected the first wall and the second wall, and a fourth wall can be opposite the third wall. A walled enclosure can be disposed between the four walls and can define a pump seat that can house the piezoelectric pump. There can be a first bore depending through the base from the pump seat on the first side of the base to the second side of the base. The first bore can be connected to an exhaust conduit that can have at least one lumen fluidly coupled to the first bore. There can be a second bore depending through the base that can be connected to an intake conduit that can have at least one lumen fluidly coupled to the second bore. The piezoelectric pump can be coupled to the first side of the base at the pump seat. The lid can be coupled to the pump casing and can be configured to seal the piezoelectric pump between the pump casing and the lid. The temperature sensor can be disposed between the pump casing and the lid. The therapy unit can further include a diaphragm pump positioned proximate to the pump module. The diaphragm pump can be configured to be in fluid communication with the tissue site.

The piezoelectric pump and the diaphragm pump can be started to draw the tissue site to a desired negative-pressure. The intake conduit can be used to draw fluid from the tissue site into the pump module. The exhaust conduit can be used to allow fluid in the pump module to escape the pump module.

The system can further include a fluid storage canister. Exudate from the tissue site can be collected in the fluid storage canister as the tissue site is being drawn to the desired negative pressure. The fluid storage canister can be configured to be coupled to a wall of the therapy unit and further configured to be in fluid communication with the pump module.

The therapy unit can further comprise a forced air device positioned proximate to the pump module. The forced air device can be turned on to regulate an internal temperature of the pump module. The temperature sensor can monitor the internal temperature of the pump module. If the internal temperature of the pump module is greater than a predetermined temperature, the forced air device can be started. The forced air device can direct a fluid flow over the lid. The fluid flow can be configured to reduce the internal temperature of the pump module. If the internal temperature is less than a predetermined temperature, the forced air device can be stopped.

The forced air device can be used to optimize fluid storage capacity of the fluid storage canister. The forced air device can be positioned so that the fluid flow can be directed into the fluid storage canister after it has traveled over the lid. The fluid flow can then be directed over a high moisture vapor transmission rate membrane located in the fluid storage canister. The fluid flow can assist with evaporation of exudate located in the fluid storage canister.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a schematic sectional view of a therapy unit that may be used with the therapy system of FIG. 1;

FIG. 3 is an exploded view of a pump module that may be associated with some embodiments of the therapy system of FIG. 1;

FIG. 4 is a perspective view of the pump module of FIG. 3;

FIG. 5 is a cross-sectional view of the pump module of FIG. 4 taken along line 5-5;

FIG. 6 is a cutaway view of a portion of a pathway enclosure, the pump module, and a forced-air device that may be associated with some embodiments of the therapy system of FIG. 1;

FIG. 7 is an exploded view of a canister that may be used with the therapy system of FIG. 1;

FIG. 8 is a schematic sectional view of the canister of FIG. 7;

FIG. 9 is a schematic sectional view of the therapy unit of FIG. 2 illustrating an operative embodiment of the therapy system of FIG. 1;

FIG. 10 is a graph illustrating a relationship between pump pressure, electrical energy consumed by a pump, and a pump temperature with respect to time that may be associated with some embodiments of the therapy system of FIG. 1;

FIG. 11 is a graph illustrating a relationship between electrical energy consumed by the pump, and the pump temperature with respect to time that may be associated with some embodiments of the therapy system of FIG. 1;

FIG. 12 is a graph illustrating the moisture vapor transmission rate of the canister at different temperatures that may be associated with some embodiments of the therapy system of FIG. 1;

FIG. 13 is a cutaway view of another embodiment of the pathway enclosure, the pump module, and the forced air device that may be associated with some embodiments of the therapy system of FIG. 1;

FIG. 14 is a detailed view of a portion of FIG. 13;

FIG. 15 is a schematic sectional view of another embodiment of the therapy unit including the pathway enclosure of FIG. 13 illustrating an operative embodiment of the therapy system of FIG. 1.

FIG. 16 is a schematic sectional view of another embodiment of the therapy unit of the therapy system of FIG. 1; and

FIG. 17 is a sectional view of the therapy unit of FIG. 16 illustrating addition details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

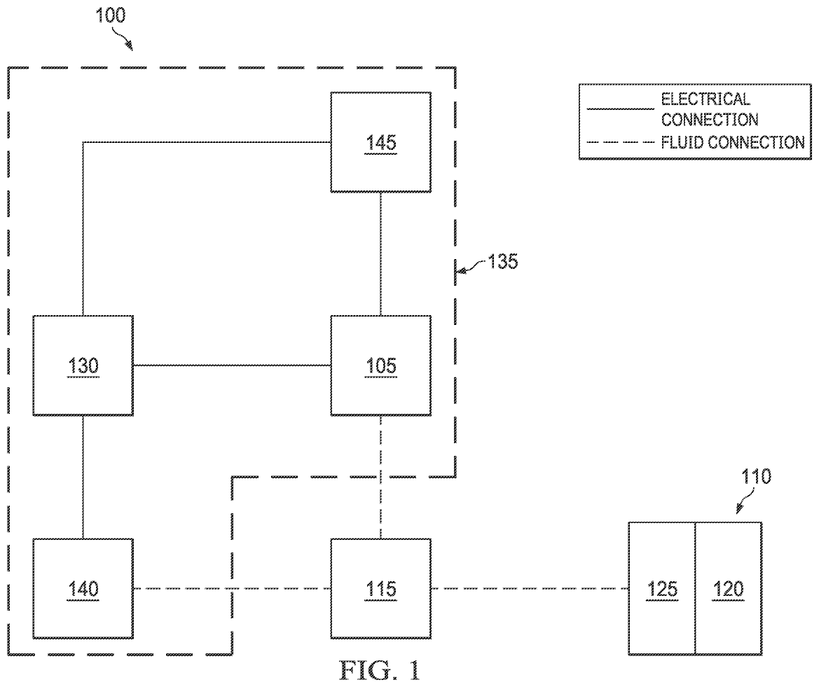

FIG. 1 is a simplified functional block diagram of an example embodiment of a therapy system 100 that can provide negative-pressure therapy to a tissue site in accordance with this specification. The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a canister 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 140 and a second sensor 145 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 135.

In general, components of the therapy system 100 may be coupled directly or indirectly. For example, the negative-pressure source 105 may be directly coupled to the canister 115 and may be indirectly coupled to the dressing 110 through the canister 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300mm Hg (−39.9 kPa).

The canister 115 is representative of a container, canister, pouch, or other storage component, which can be used to manage exudates and other fluids withdrawn from a tissue site. In many environments, a rigid canister may be preferred or required for collecting, storing, and disposing of fluids. In other environments, fluids may be properly disposed of without rigid canister storage, and a re-usable canister could reduce waste and costs associated with negative-pressure therapy.

A controller or a control board, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 140 and the second sensor 145, are generally known in the art as any apparatus operable to detect or measure a physical phenomenon or property, and generally provide a signal indicative of the phenomenon or property that is detected or measured. For example, the first sensor 140 and the second sensor 145 may be configured to measure one or more operating parameters of the therapy system 100. In some embodiments, the first sensor 140 may be a transducer configured to measure pressure in a pneumatic pathway and convert the measurement to a signal indicative of the pressure measured. In some embodiments, for example, the first sensor 140 may be a piezo-resistive strain gauge. The second sensor 145 may optionally measure operating parameters of the negative-pressure source 105, such as a voltage or current, in some embodiments. Preferably, the signals from the first sensor 140 and the second sensor 145 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™M dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™M dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in canister 115.

In some embodiments, the controller 130 may receive and process data from one or more sensors, such as the first sensor 140. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

Negative-pressure therapy has been repeatedly shown to be effective in the treatment of difficult to heal wounds. Unfortunately, some negative-pressure sources generate noise which can dissuade patients from complying with treatment. Piezoelectric pumps have been used in some negative-pressure systems to address these noise concerns. A piezoelectric pump may be capable of operation in the high frequency range. As used herein, a high frequency range is a frequency range beyond the range of frequencies detectable by the human car, e.g., a frequency greater than 16 kilohertz (kHz). As well as producing minimal noise, piezoelectric pumps are small and come at a low cost. Despite these benefits, piezoelectric pumps that operate in the high frequency range may have difficulty generating sufficient negative pressure for negative-pressure therapy. Maintaining the target pressure level in a negative pressure environment is a demanding application for a piezoelectric pump. To use a piezoelectric pump to generate negative-pressure, the piezoelectric pump may operate continuously or semi-continuously. Continuous or semi-continuous operation of a piezoelectric pump can cause significant heat built up within the piezoelectric pump.

In some environments, piezoelectric pumps may be considered less efficient than diaphragm pumps commonly used to generate negative pressure. Efficiency of a pump may relate the amount of energy consumed to produce a desired output from the pump. For example, where a pump is used to generate negative pressure, the efficiency of the pump refers to the amount of electric energy consumed by the pump to produce a particular negative pressure. A pump that has a lower efficiency than another pump will generally require more electrical energy to produce the same pneumatic output. The additional electrical energy used is generally converted to heat as a by-product of the pumping process. Thus, where a piezoelectric pump is operated continuously or semi-continuously for negative pressure, the lower relative efficiency leads to higher operating temperatures and significant heat buildup. For a piezoelectric pump, the heat generated by the pumping process has often limited the use of piezoelectric pumps due to concerns about patient exposure to excess temperatures. Furthermore, an excess buildup of heat within a piezoelectric pump can potentially degrade the operation of the piezoelectric pump, decreasing the pump life. A negative-pressure system capable of controlling the temperature at which the piezoelectric pump operates while also utilizing the excess heat energy from the piezoelectric pump could address a long-felt need in the art.

These limitations and others may be addressed by the therapy system 100, which can have an overall noise level that is lower than known systems, in a smaller therapy unit than in other known systems, while also having a reduced weight. Still further, the therapy system 100 can control the temperature of the device to gain the full performance of the pumps used in the system. The therapy system 100 can also scavenge heat energy from the system to improve the efficiency of the negative-pressure source, increase battery life or conversely reduce battery capacity for similar effect, improve fluid handling, and decrease the number of canister changes, reducing the environmental impact of the therapy system 100 over other known therapy systems.

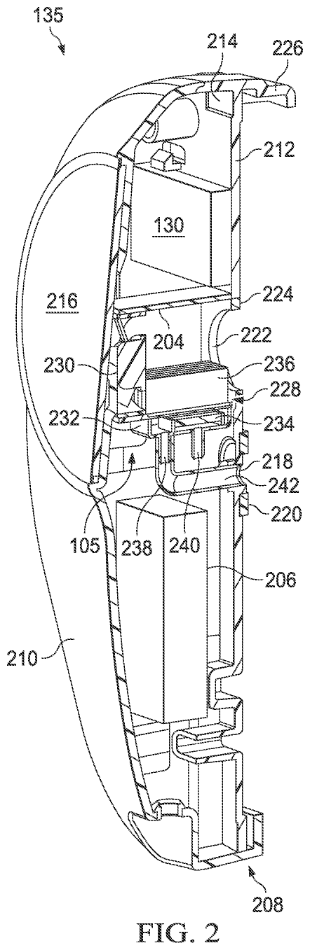

FIG. 2 is a perspective cutaway view illustrating additional details that may be associated with some embodiments of the therapy unit 135. In some embodiments, the therapy unit 135 may comprise the negative-pressure source 105, a pathway enclosure 204, a power source 206, and the controller 130 all disposed within a housing 208.

The housing 208 can be an oblong body having a first wall 210 and a second wall 212 coupled to each other to form an interior space 214. The first wall 210 may be an oblong body having a generally convex shaped exterior facing away from the second wall 212 and a concave shaped interior facing the second wall 212. The first wall 210 and the second wall 212 may be formed from a plastic, thermoplastic, thermoset, fiber-type material, ceramic, metal, or other material that is capable of maintaining a desired shape and that is capable of being exposed to wound fluids or other liquids. The first wall 210 and the second wall 212 may be adhesively bonded, welded, or attached in any other suitable manner. Preferably, the means of attachment will provide a substantially gas impermeable seal between the first wall 210 and the second wall 212 such that air flow within the interior space 214 may be manipulated through the use of internally mounted barriers, walls, or other devices.

In some embodiments, the first wall 210 can have a control panel 216 coupled to the exterior. The control panel 216 can comprise one or more user interfaces configured to permit a user to interact with the controller 130. In some embodiments, the control panel 216 may include a graphical user interface, a touchscreen, and/or one or more motion tracking devices. For instance, the control panel 216 may also include one or more display screens, such as a liquid crystal display (“LCD”), lighting devices, such as light emitting diodes (“LED”) of various colors, and audible indicators, such as a whistle, configured to emit a sound that may be heard by an operator. Additionally, in some embodiments the control panel 216 may further include one or more devices, such as knobs, buttons, keyboards, remotes, touchscreens, ports that may be configured to receive a discrete or continuous signal from another device, or other similar devices, which may be configured to permit the external environment to interact with the user interface. For example, the control panel 216 may permit the external environment, for example, a user within the external environment, such as a physician, care-giver, or patient, to select a therapy having a particular characteristic, for example, to be performed by the therapy system 100. In some embodiments, the control panel 216 may display information to the external environment such as a therapy duration, a type of therapy, or an amount of negative pressure being supplied, for example.

The second wall 212 may be substantially planar and may have a negative-pressure inlet, such as a fluid inlet 218. The fluid inlet 218 can be an opening through the second wall 212 permitting fluid communication across the second wall 212. In some embodiments, a seal 220 can be coupled to the second wall 212. The seal 220 can surround the fluid inlet 218. The seal 220 can be formed from a material configured to form a seal between the second wall 212 and an object abutting the second wall 212 at the fluid inlet 218. In some embodiments, the seal 220 may be rubber, silicone, or other materials capable of forming a gas impermeable seal between the second wall 212 and an object abutting the second wall 212.

The second wall 212 may also have a positive-pressure outlet, such as a fluid outlet 222. The fluid outlet 222 can be an opening through the second wall 212 permitting fluid communication across the second wall 212. In some embodiments, the fluid outlet 222 may be larger than the fluid inlet 218. In some embodiments, a seal 224 can be coupled to the second wall 212. The seal 224 can surround the fluid outlet 222. The seal 224 can be formed from a material configured to form a seal between the second wall 212 and an object abutting the second wall 212 at the fluid outlet 222. In some embodiments, the seal 224 can be rubber, silicone, or other materials capable of forming a gas impermeable seal with the second wall 212 and an object abutting the second wall 212.

The second wall 212 can also include one or more attachment points 226. The attachment points 226 may be a device, such as a latch or catch, permitting another object to be coupled to therapy unit 135 at the second wall 212. In some embodiments, the attachment points 226 may be formed from a same or similar material as the second wall 212. In other embodiments, the attachment points 226 may be formed from any material configured to provide a secure point of attachment for a secondary object, such as the canister 115.

The pathway enclosure 204 may be positioned within the interior space 214 to segregate a volume of the interior space from the remainder of the interior space 214. Preferably, the pathway enclosure 204 may be a physical barrier within the interior space 214 that directs fluid flow. A first end of the pathway enclosure 204 may be proximate to the first wall 210. In some embodiments, the first end of the pathway enclosure 204 may be sealed to the first wall 210. In other embodiments, the first end of the pathway enclosure 204 may permit fluid flow between the first wall 210 and the first end of the pathway enclosure 204 so that fluid may flow into the volume segregated by the pathway enclosure 204. In some embodiments, a second end of the pathway enclosure 204 can be proximate to the fluid outlet 222 of the second wall 212. The path way enclosure 204 may direct a fluid flow within the interior space between the first wall 210 and the second wall 212. In some embodiments, the second end of the pathway enclosure 204 may couple to the second wall 212 of the therapy unit 135. For example, the second end of the pathway enclosure 204 may be sealed to the second wall 212 at the fluid outlet 222 so that fluid flowing from the first end of the pathway enclosure 204 to the second end of the pathway enclosure 204 may flow from the volume of the pathway enclosure 204 through the fluid outlet 222, exiting the interior space 214. In some embodiments, the pathway enclosure 204 may be formed from the same or similar material as the first wall 210 and the second wall 212. For example, the pathway enclosure 204 may be formed from a plastic, thermoplastic, thermoset, fiber-type material, ceramic, metal, or other material that is capable of maintaining a desired shape and that is capable of being exposed to wound fluids or other liquids. The pathway enclosure 204 may be coupled to the first wall 210 and the second wall 212 of the housing 208 by adhesively bonding, welding, or attaching in any other suitable manner.

In some embodiments, the negative-pressure source 105 may be disposed in the interior space 214. The negative-pressure source 105 may comprise a pump module 228 and a forced-air device 230. In other embodiments, the negative-pressure source 105 may further include a secondary pump such as a diaphragm pump. In some embodiments, the pump module 228 includes a pump casing 232, a pump 234, and a lid 236. The pump casing 232 may have an intake, such as an intake 238, and an exhaust, such as an exhaust 240. The intake 238 may be a fluid port configured to permit fluid to enter the pump module 228, and the exhaust 240 may be a fluid port configured to permit fluid to exit the pump module 228. In some embodiments, a fluid conductor, such as an intake conduit 242, may fluidly couple the pump module 228 to an exterior of the therapy unit 135. For example, the intake conduit 242 may have a first end coupled to the second wall 212 at the fluid inlet 218. A second end of the intake conduit 242 may be fluidly coupled to the intake 238, providing a fluid path from the fluid inlet 218 to the intake 238.

In some embodiments, the exhaust 240 can be a fluid port coupling an exhaust of the pump 234 to an exterior of the pump module 228. For example, the exhaust 240 may fluidly couple the exhaust of the pump 234 to the interior space 214. In other embodiments, the exhaust 240 may be fluidly coupled to an exterior of the therapy unit 135. For example, a fluid conductor may fluidly couple the exhaust 240 to the fluid outlet 222 or a vent located in the first wall 210 or the second wall 212.

The pump 234 can be disposed between and fluidly sealed to the pump casing 232 and the lid 236. In some embodiments, a pump outlet of the pump 234 may be fluidly coupled to the exhaust 240, and a pump inlet may be fluidly coupled to the intake 238. The lid 236 can be coupled to the pump casing 232. In some embodiments, the lid 236 may seal the pump 234 inside the pump casing 232 preventing fluid flow into and out of the pump module 228 except by the intake 238 and the exhaust 240.

In some embodiments, the pump module 228 can be disposed within the interior space 214 so that the lid 236 defines at least a portion of the pathway enclosure 204. For example, in some embodiments, the pathway enclosure 204 may be an annular wall. A portion of the annular wall may be removed and replaced with the lid 236.

The forced-air device 230 may be positioned adjacent to the pump module 228. In some embodiments, the forced-air device 230 may be an axial fan configured to create a fluid flow over the lid 236 of the pump module 228. For example, the forced-air device 230 may be configured to generate a fluid flow across the lid 236 from the first end of the pathway enclosure 204 to the second end of the pathway enclosure 204. In some embodiments, the fluid flow may draw heat from the lid 236 and away from the pump module 228 using convection cooling.

The power source 206 and the controller 130 can be coupled, for example, by electric coupling or communicative coupling, to the pump module 228 and the forced-air device 230. In some embodiments, the power source 206 may be a battery or other source of electric potential that can provide electrical power to the therapy unit 135. The controller 130 can be configured to operate the pump module 228 and the forced-air device 230 for operation of the therapy unit 135.

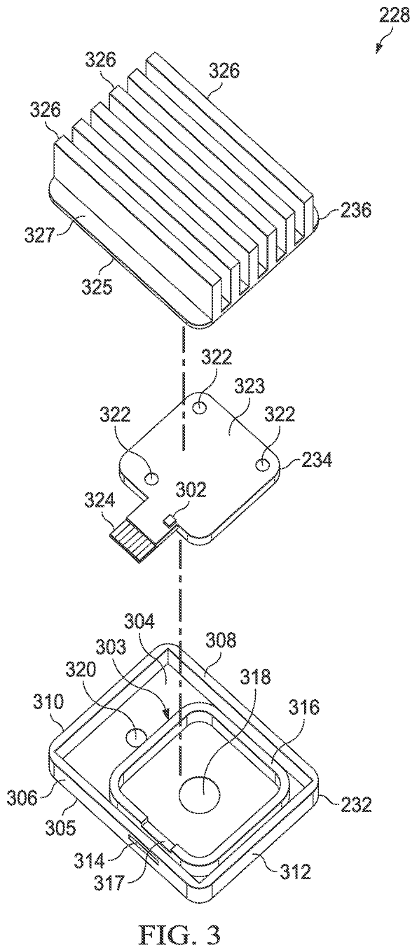

FIG. 3 is an exploded view of the pump module 228, illustrating additional details that may be associated with some embodiments. In some embodiments, the pump module 228 may include features that enable regulation of the internal temperature of the pump 234. While the figures may illustrate exemplary embodiments of the pump module 228, other exemplary pump modules 228 may have other sizes, shapes, and/or configurations.

In some embodiments, the pump module 228 may include the pump casing 232, the pump 234, the lid 236, and a temperature sensor 302. The pump casing 232 may have a base 304 with a first side 303 and a second side 305. The pump casing 232 may also include a first wall 306 protruding from the first side 303 of the base 304, and a second wall 308 opposite the first wall 306. The pump casing 232 may further include a third wall 310 perpendicular to and extending from the first wall 306 to the second wall 308, and a fourth wall 312 perpendicular to and extending from the first wall 306 to the second wall 308 opposite the third wall 310. The first wall 306 may contain an opening 314. The opening 314 may extend through the first wall 306 permitting communication across the first wall 306 from an interior of the pump casing 232 to an exterior of the pump casing 232. In some embodiments, the opening 314 may be a recess into the first wall 306 toward the first side 303. In other embodiments, the opening 314 may be surrounded by the first wall 306.

An interior wall, such as a pump seat 316, may be disposed in the interior of the pump casing 232, extending from the first side 303. In some embodiments, the pump seat 316 may have a height less than a height of the first wall 306. In other embodiments, the pump seat 316 may have a height substantially equal to a height of the first wall 306. In some embodiments, the pump seat 316 may be disposed inboard of the first wall 306, the second wall 308, the third wall 310, and the fourth wall 312. The pump seat 316 may be an annular wall having portions parallel to the first wall 306, the second wall 308, the third wall 310, and the fourth wall 312. In some embodiments, the pump seat 316 is substantially square in shape. In other embodiments, the pump seat 316 may be any other shape that is configured to receive the pump 234. For example, in some embodiments the pump seat 316 may be circular, rectangular, triangular, or an amorphous shape. In some embodiments, the pump seat 316 may be configured to receive the pump 234 inside the pump module 228. The pump seat 316 may include a recess 317 that is substantially aligned in size and shape with the opening 314.

The pump casing 232 may further comprise a first bore 318 depending into and through the base 304 of the pump casing 232 from the first side 303 to the second side 305. The first bore 318 may be located in a space bounded by the pump seat 316 and the base 304. The pump casing 232 may also comprise a second bore 320 depending into and through the base 304 of the pump casing 232 from the first side 303 to the second side 305. The second bore 320 may be located within a space bounded by the base 304, the first wall 306, the second wall 308, and the third wall 310. In other embodiments, the second bore 320 may be located in other areas provided the second bore 320 does not fluidly communicate with the first bore 318.

The pump casing 232 may be comprised of an insulating material. The insulating material may be any material with a relatively low thermal conductivity. For example, a thermal conductivity less than 1.0 watts per meter-Kelvin (W/mK). In some embodiments a relatively low thermal conductivity can be less than 0.5 W/mK, 0.026 W/mK, or less. The lower the thermal conductivity of the pump casing 232, the easier the route will be for any excess heat from the pump 234 to travel to the lid 236 of the pump module 228. In some preferred embodiments, the insulating material may be thermoplastic, foam, or another similar material. In some embodiments, the insulating material may be a vacuum wall. A vacuum wall can be a pneumatically sealed chamber having air within the chamber removed. For example, the first wall 306 can be a hollow body having an internal chamber having the air within the internal chamber removed and vacuum sealed. Similarly, the second wall 308, the third wall 310, the fourth wall 312, and the pump seat 316 may be vacuum walls.

The pump 234 may be coupled to the pump seat 316. In some embodiments, the pump 234 may be sealed to an end of the pump seat 316 opposite the base 304, creating a sealed space between the base 304, the pump seat 316, and the pump 234. The pump 234 may have a pump inlet 322 and a pump outlet. In some embodiments, the pump inlet 322 can be three pump inlets 322. The pump inlets 322 can be equidistantly spaced from each other on a surface of the pump 234. In other embodiments, the pump inlets 322 can be grouped on the pump 234 to preferentially generate a negative pressure at a particular location. In some embodiments the pump inlets 322 can be fluidly coupled to the intake conduit 242 of the therapy unit 135.

The pump 234 may have a generally square shaped pump body with a flat face 323 facing away from a printed circuit board. The pump inlets 322 can be disposed in the flat face 323. In some embodiments, the pump 234 may have dimensions of about 21 mm by about 19 mm by about 3.4 mm, each having a tolerance of about 0.2 mm. The pump 234 may have a free flow rate at 18 Vdc of greater than or equal to 155 ml/min and a static pressure at about 19.5 Vdc of greater than or equal to about 50kPa at an ambient temperature of about 18 to 28 degrees Celsius, a relative humidity of 25% to about 85% relative humidity, and an atmospheric pressure between about 950 hPa and 1020 hPa.

In some embodiments, the pump 234 can include one or more electrical contacts, such as an electrical connection 324. The electrical connection 324 can project from the pump 234. In some embodiments, the electrical connection 324 can provide a coupling to supply electrical potential to the pump 234. The pump 234 may have a low pneumatic efficiency and may have heat as a by-product. In some preferred embodiments, the pump 234 may be a piezoelectric pump. For example, the pump 234 may be a Murata Microblower MZB3004T04 available from Murata Manufacturing Co., Ltd. of Kyoto, Japan.

In some embodiments, the temperature sensor 302 can be coupled to the flat face 323 of the pump body of the pump 234 proximate to the electrical connection 324. The temperature sensor 302 can be configured to provide a signal representing a temperature at the flat face 323 of the pump 234 through the electrical connection 324. In some embodiments, the signal generated by the temperature sensor 302 may approximate the temperature of the pump module 228.

The lid 236 may be configured to be coupled to the pump casing 232. In some embodiments, the lid 236 may have a first surface 325 and a second surface 327. The first surface 325 of the lid 236 may be a generally flat surface and be configured to be coupled to the first wall 306, the second wall 308, the third wall 310, and the fourth wall 312. In some embodiments, the first surface 325 of the lid 236 may seal to the first wall 306, the second wall 308, the third wall 310, and the fourth wall 312 to form a sealed space between the lid 236, the base 304, the first wall 306, the second wall 308, the third wall 310, and the fourth wall 312.

The second surface 327 of the lid 236 may contain a plurality of ribs 326 extending away from the pump 234. The plurality of ribs 326 may have a length equal to a length of the lid. In some embodiments, the length can be about equal to a distance from the third wall 310 to the fourth wall 312. In some embodiments, the ribs 326 may be parallel to the first wall 306 and the second wall 308 of the pump casing 232. In other embodiments, the ribs 326 may be parallel to the third wall 310 and the fourth wall 312 of the pump casing 232. In still other embodiments, the ribs 326 may be disposed at a non-normal angle to the first wall 306, the second wall 308, the third wall 310, and the fourth wall 312. The plurality of ribs 326 can be selected to maximize surface area of the plurality of ribs 326 for the space in which the lid 236 is placed. In some embodiments, the lid 236 may be formed from a thermally conductive material. In some preferable embodiments, the lid 236 may be formed from a material of relatively high thermal conductivity such as aluminum, copper, or another similar material. In some embodiments, a high thermal conductivity can be greater than 200 W/mK, for example 247 W/mK or 398 W/mK. In some embodiments, the lid 236 of the pump module 228 operates as a heat sink, conducting heat away from the pump module 228. Heat generated by operation of the pump 234 may be conducted though the lid 236 to distal ends of the plurality of ribs 326. There, convection cooling may transfer heat from the lid 236 to the interior space 214 of the therapy unit 135. The lid 236 may harvest the heat from the pump 234 and direct the heat away from the pump 234 through the plurality of ribs 326 of the lid 236.

In some embodiments, the insulating material of the pump casing 232 may retain some heat proximate to the pump 234. In some embodiments, an increase in the temperature in the pump module 228 may increase the efficiency of the pump 234. For example, the pump may convert more of the electrical energy received into fluid flow as the temperature of the pump 234 and the pump module 228 increases. In some embodiments, as the temperature of the pump module 228 approaches about 50 degrees Celsius, the pump 234 may operate most efficiently.

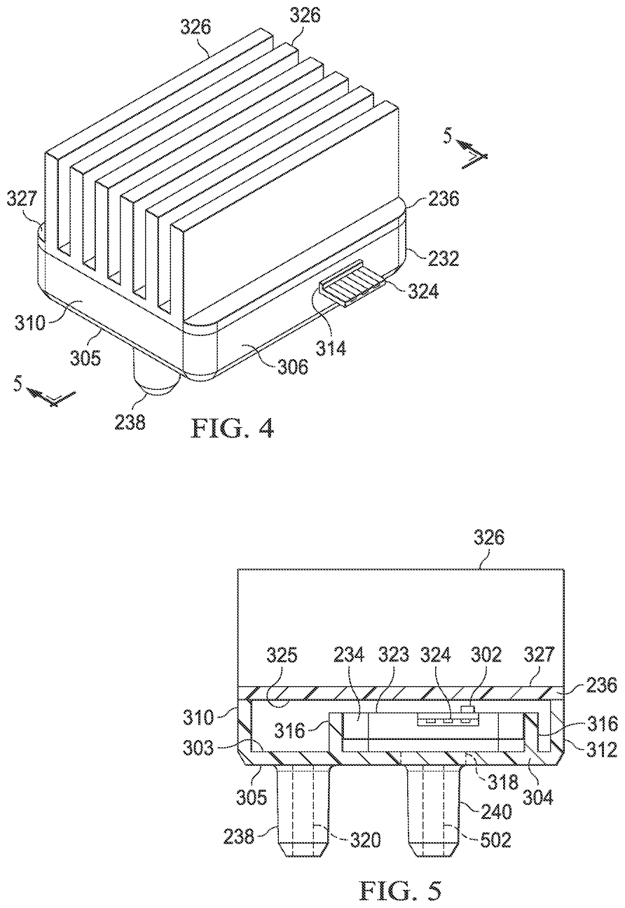

FIG. 4 is a perspective view illustrating additional details that may be associated with some embodiments of the pump module 228 of FIG. 3. In some embodiments, the electrical connection 324 can be inserted through the opening 314. Preferably, the opening 314 can be sized to accommodate the electrical connection 324 while sealing to the electrical connection 324. In some embodiments, a seal, such as a grommet, adhesive, or other sealing device can be disposed at the opening 314 to seal the electrical connection 324 to the first wall 306.

The lid 236 can be coupled to the pump casing 232. In some embodiments, the lid 236 can be adhered, welded, bonded, or otherwise attached to the first wall 306, the second wall 308, the third wall 310, and the fourth wall 312. The lid 236 can be sealed to the pump casing 232 to create a sealed space containing the pump 234.

FIG. 5 is a cross sectional view of the pump module 228 taken along line 5-5 of FIG. 4. The pump 234 can be coupled to the pump seat 316. In some embodiments, the pump 234 can be adhered to the pump seat 316. The pump 234 may be sealed to the pump seat 316. In some embodiments, the fluid outlet of the pump 234 extends from the pump body opposite the flat face 323. In some embodiments, the fluid outlet of the pump 234 may be a jet nozzle extending about 4.5 mm from the surface of the pump 234. In some embodiments, the fluid outlet of the pump 234 may be the exhaust 240 having a lumen 502 extending through the exhaust 240. In some embodiments, the exhaust 240 of the pump 234 may be a fluid coupling such as a port or nipple having a central passage or lumen configured to be fluidly coupled to a tube or other device. In some embodiments, the exhaust 240 is integral to the pump 234 and inserts into the pump casing 232 through the first bore 318. Fluid may pass from the interior of the pump module 228 to an exterior of the pump module 228 through the first bore 318 and the exhaust 240.

In some embodiments, the intake 238 may project from the second side 305 of the base 304. The intake 238 can be a port or nipple having a central passage or lumen configured to be fluidly coupled to a tube or other device. In some embodiments, the intake 238 can be fluidly coupled to the intake conduit 242 by inserting the intake 238 into a lumen of the intake conduit 242. The intake 238 may be fluidly coupled to the second bore 320, and the intake 238 may be in fluid communication with the pump inlets 322 of the pump 234 through the second bore 320. Operation of the pump 234 may draw fluid into the pump module 228 through the intake 238 and force fluid from the pump module 228 through the exhaust 240.

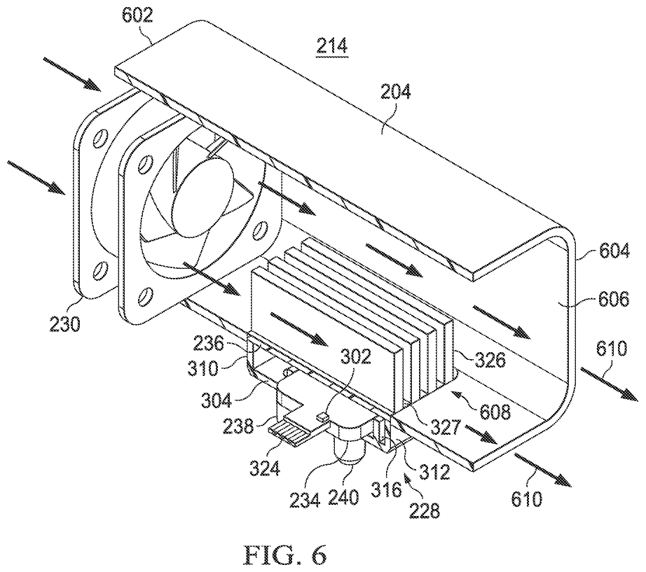

FIG. 6 is a perspective cut-away view of a portion of the therapy unit 135 illustrating additional details that may be associated with some embodiments. The pathway enclosure 204 may have a first end 602 and a second end 604. The first end 602 of the path way enclosure 204 can be adjacent to the first wall 210, and the second end 604 of the pathway enclosure 204 can be disposed adjacent the fluid outlet 222 of the second wall 212. In some embodiments, the pathway enclosure 204 may have a square cross-sectional profile having rounded corners. In other embodiments, the pathway enclosure 204 can have a circular, triangular, or amorphous cross-section. In some embodiments, the pathway enclosure 204 may define a volume 606 within the interior space 214. The volume 606 may be partially fluidly isolated from the interior space 214. In other embodiments, the volume 606 may be fully fluidly coupled to the interior space 214.

The forced-air device 230 can be positioned in the volume 606 adjacent to the first end 602. The forced-air device 230 can be an axial fan, a centrifugal fan, a cross-flow fan, a bellows, a coanda effect device, a convective airflow device, an electrostatic airflow device, or other similar device configured to generate a fluid flow. In some embodiments, the pathway enclosure 204 may surround the forced-air device 230, generally directing the fluid flow generated by the forced-air device from the first end 602 to the second end 604 of the pathway enclosure 204 while maintaining the fluid flow within the volume 606.

An opening 608 can be disposed in at least one wall of the pathway enclosure 204. In some embodiments, the opening 608 can be sized to receive at least a portion of the pump module 228. For example, the opening 608 can be sized so that the lid 236 can be inserted into the volume 606 of the pathway enclosure 204. In some embodiments, the second surface 327 of the lid 236 can be flush with a surface of the at least one wall of the pathway enclosure 204. The plurality of ribs 326 can project from the second surface 327 of the lid 236 into the volume 606 of the pathway enclosure 204. In some embodiments, the forced-air device 230 can be oriented relative to the lid 236 to permit the forced-air device 230 to generate a fluid flow 610 parallel to a length of each of the plurality of ribs 326. In the illustrative embodiment, the forced-air device 230 may be an axial fan oriented so that an axis of rotation of the blades forming the forced-air device 230 is parallel to the plurality of ribs 326.

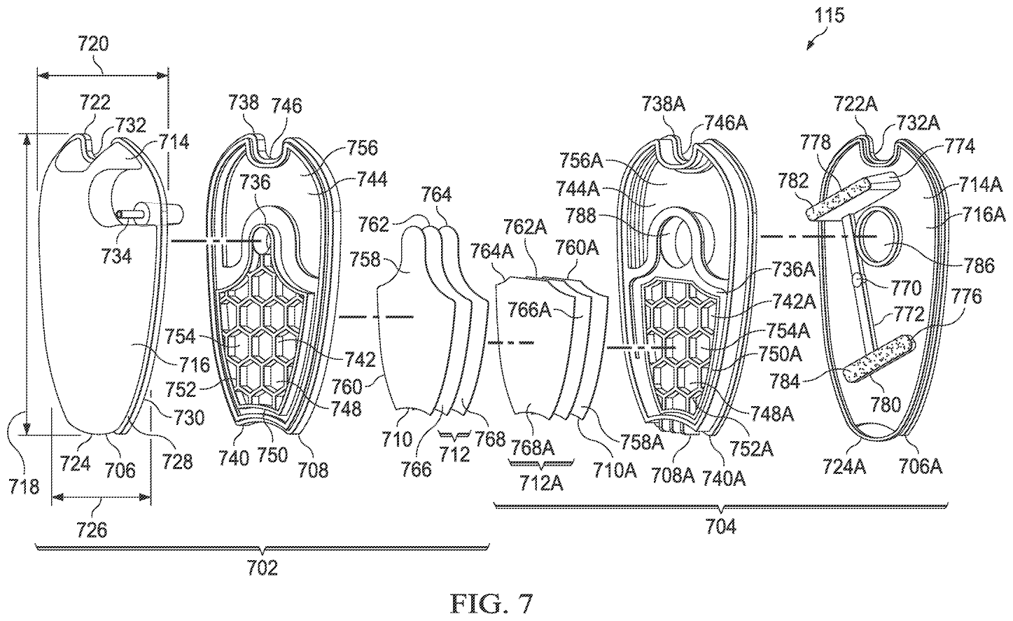

FIG. 7 is an exploded view of the canister 115, illustrating additional details that may be associated with some embodiments of the therapy system 100. In some embodiments, the canister 115 may comprise a first section, such as a first canister section 702, and a second section, such as a second canister section 704, both being configured to store fluid. The first canister section 702 may be fluidly connected to a dressing, such as the dressing 110. The second canister section 704 may be configured to be fluidly connected to a negative-pressure source, such as the negative-pressure source 105. The canister 115 may further comprise an airflow pathway. The airflow pathway may be disposed between the first canister section 702 and the second canister section 704. The airflow pathway may be configured to allow transmission of evaporated fluids from the canister 115 to the ambient environment.

The first canister section 702 may comprise an outer wall, an exterior wall, a first wall, or a first outer section 706, a carrier or a first filter carrier 708, a distribution layer or a first nonwoven layer 710, and one or more evaporative layers or a first evaporative section 712. In some embodiments, the first outer section 706 may have a base 714 with a first surface 716 and a second surface opposite the first surface 716. The first surface 716 may have a generally convex shape and face away from the second canister section 704. The second surface may be concave shaped and face the second canister section 704. In the illustrative embodiment, the base 714 may have a generally ovular shape having a flattened end. In other embodiments, the base 714 may be rectangular, circular, triangular, ovular, or amorphous in shape. The base 714 may have a length 718, and a width 720 at a first end 722, the width 720 may be perpendicular to the length 718. There may be a second end 724 opposite the first end 722. The second end 724 may have a width 726. In some embodiments, the width 720 may be greater than the width 726. In other embodiments, the width 720 may be substantially equal to the width 726. In some embodiments, the length 718 may be greater than the width 720 and the width 726. In other embodiments, the length 718 may be substantially equal to the width 720 and the width 726.

The base 714 may have a periphery or exterior edge 728. An extension 730 may extend from the exterior edge 728 of the base 714 towards the second canister section 704. The extension 730 may be coincident with the exterior edge 728 of the base 714. The extension 730 may extend from and be perpendicular to a plane defined by the exterior edge 728 of the base 714. In some embodiments, the exterior edge 728 may be a joint of the extension 730 and the base 714. For example, the exterior edge 728 may be formed by coupling the extension 730 to the base 714. In some embodiments, the exterior edge 728 may comprise a chamfered beveled edge. In other embodiments, the exterior edge 728 may comprise a vertex of a perpendicular angle between the extension 730 and the base 714. In some embodiments, a receiver or a notch 732 can be formed in the first end 722. In some embodiments, the notch 732 extends through the base 714 from the first surface 716 to the second surface. The notch 732 may be positioned proximate a center of the width 720. In other embodiments, the notch 732 may not be centered on the width 720. In some embodiments, the notch 732 may have a width less than the width 720.

A fluid inlet 734 can be disposed in the first outer section 706. In some embodiments, the fluid inlet 734 may provide fluid communication across the base 714. In some embodiments, the fluid inlet 734 may be configured to fluidly couple the canister 115 to the dressing 110. For example, a fluid conductor may be coupled to the fluid inlet 734 and similarly coupled to the dressing 110 to fluidly couple the canister 115 to the dressing 110. In some embodiments, the fluid inlet 734 may be disposed proximate the first end 722 of the base 714. In some embodiments, the fluid inlet 734 may be positioned near a midpoint of the width 720 at the first end 722. In the illustrative embodiment, the fluid inlet 734 may be off center on the width 720 of the first end 722. In other embodiments, the fluid inlet 734 may be disposed in other locations on the base 714.

The first filter carrier 708 may comprise a first surface 736 and a second surface opposite the first surface 736. The first filter carrier 708 may have a first end 738 and a second end 740 opposite the first end 738. The first end 738 may have a width substantially equal to the width 720 of the first outer section 706, and the second end 740 may have a width substantially equal to the width 726 of the first outer section 706. In other embodiments, the first end 738 may have a width less than the width 720, and the second end 740 may have a width less than the width 726. The first filter carrier 708 may have a length that is substantially equal to the length 718 of the first outer section 706.

The first surface 736 of the first filter carrier 708 may be facing the first outer section 706 and the second surface of the first filter carrier 708 may be facing the first nonwoven layer 710. The first filter carrier 708 may comprise a first portion 742 and a second portion 744. The first portion 742 may extend from the second end 740 to the second portion 744. The second portion 744 may extend from the first portion 742 to the first end 738.

In some embodiments, a receiver or a notch 746 can be formed in the first end 738. In some embodiments, the notch 746 extends through the first filter carrier 708 from the first surface 736 to the second surface. The notch 746 may be positioned proximate to a center of the width of the first end 738. In other embodiments, the notch 746 may not be centered on the width of the first end 738. In some embodiments, the notch 746 may have a width less than the width of the first end 738. In some embodiments, the notch 746 may be aligned or coincident with the notch 732 of the first outer section 706.

The first portion 742 may have a periphery or a peripheral portion, such as a first filter boundary 750 surrounding and defining an opening 748. In some embodiments, the opening 748 can comprise a substantial portion of the first portion 742. For example, the opening 748 may comprise greater than 50% of the surface area of the first portion 742. In some embodiments, the opening 748 may comprise about 50% to 70% of the surface area of the first portion 742. In other embodiments, the opening 748 may be about 90% or greater of the surface area of the first portion 742.

A first support framework, such as a first filter section 752 can be disposed in the opening 748. The first filter section 752 can comprise a plurality of arms, beams, or braces extending across the opening 748. In some embodiments, the first filter section 752 forms a plurality of holes 754. Each hole 754 of the plurality of holes 754 may have a hexagonal shape. In some embodiments, each vertex of each hole 754 may be proximate to at least one vertex of an adjacent hole 754. In other embodiments, the plurality of holes 754 may comprise different sizes and shapes. The plurality of holes 754 may maintain all or substantially all of the opening 748 of the first portion 742. The plurality of holes 754 of the first filter section 752 may be configured to allow fluid flow across the first filter section 752.

An opening 756 can be disposed in the second portion 744. The opening 756 can be configured to provide fluid communication across the first filter carrier 708 through the second portion 744. In some embodiments, the opening 756 can comprise a substantial portion of the second portion 744. For example, the opening 756 may comprise greater than 50% of the surface area of the second portion 744. In other embodiments, the opening 756 may be about 90% or greater of the surface area of the second portion 744.

The periphery of the first surface 736 of the first filter carrier 708 may be coupled to the extension 730 of the first outer section 706. In other embodiments, the first filter carrier 708 may comprise a different size or shape but may still couple to the first outer section 706 to form the structure of the first canister section 702. In some embodiments, the first filter carrier 708 may be coupled to the first outer section 706 at one or more attachment points. In other embodiments, the first filter carrier 708 and the first outer section 706 may be coupled by compression gaskets, adhesives, a weld, or any other suitable method of coupling to seal the first filter carrier 708 to the first outer section 706.

The first nonwoven layer 710 may have a first surface 758 and a second surface opposite the first surface 758. The first nonwoven layer 710 may be substantially the same shape as the first portion 742 of the first filter carrier 708. The first nonwoven layer 710 may be smaller in size than the first portion 742 of the first filter carrier 708 such that a periphery 760 of the first nonwoven layer 710 aligns with the first filter boundary 750 of the first filter carrier 708. The first surface 758 of the first nonwoven layer 710 may be configured to cover the opening 748 of the first filter carrier 708. The first surface 758 of the first nonwoven layer 710 may be coupled to the second surface of the first filter carrier 708 along the first filter boundary 750. The first nonwoven layer 710 may contain additives that allow fluids from the canister 115 to be distributed to the first evaporative section 712.

The first evaporative section 712 may comprise one or more evaporative membrane layers such as a first evaporative layer 762 and a second evaporative layer 764. The first evaporative layer 762 may comprise a first surface 766 and a second surface opposite the first surface 766. The second evaporative layer 764 may have a first surface 768 and a second surface opposite the first surface 768. The first evaporative layer 762 and the second evaporative layer 764 may be substantially the same size and shape as the first nonwoven layer 710. The first surface 766 of the first evaporative layer 762 may be configured to couple to the second surface of the first nonwoven layer 710. The first surface 768 of the second evaporative layer 764 may be configured to couple to the second surface of the first evaporative layer 762. In some embodiments, the first nonwoven layer 710, the first evaporative layer 762, and the second evaporative layer 764 may be welded to the first filter carrier 708 along the first filter boundary 750. In other embodiments, the first nonwoven layer 710, the first evaporative layer 762, the second evaporative layer 764, and the first filter carrier 708 may be coupled by other methods such as adhesives, compression gaskets, or other attachment methods.

The canister 115 may have a second section such as second canister section 704. The second canister section 704 may comprise an outer wall, an exterior wall, a second wall or a second outer section 706A, a carrier or a second filter carrier 708A, a distribution layer or a second nonwoven layer 710A, and an evaporative layer or a second evaporative section 712A. The second outer section 706A may have a base 714A that is ovular in shape. Other embodiments of the second outer section 706A may be substantially circular, triangular, rectangular, or amorphous in shape. The base 714A may have a first surface 716A and a second surface opposite the first surface 716A. The base 714A may have a length substantially equal to length 718 of the first outer section 706. The base may have a first end 722A with a width substantially equal to width 720 and a second end 724A opposite the first end 722A with a width substantially equal to width 726. In some embodiments, the width of the first end 722A may be greater than the width of the second end 724A. In other embodiments, the width of the first end 722A may be substantially equal to the width of the second end 724A. In some embodiments, the length may be greater than the width of the first end 722A and the width of the second end 724A. In other embodiments, the length may be substantially equal to the width of the first end 722A and the width of the second end 724A.

In some embodiments, a receiver or a notch 732A may be formed in the first end 722A. In some embodiments, the notch 732A extends through the base 714A from the first surface 716A to the second surface. The notch 732A may be positioned proximate to a center of the width of the first end 722A. In other embodiments, the notch 732A may not be centered on the width of the first end 722A. In some embodiments, the notch 732A may have a width less than the width of the first end 722A.

A reduced pressure inlet 770 may be disposed in the second outer section 706A. The reduced pressure inlet 770 may be substantially centered with respect to the length of the second outer section 706A but may be off-centered with respect to the width of the second outer section 706A. In other embodiments, the reduced pressure inlet 770 may be substantially centered with respect to both the length and the width or may be located in another area in the second outer section 706A. In some embodiments, the reduced pressure inlet 770 may be configured to fluidly couple the canister 115 to the negative-pressure source 105. For example, a fluid conductor may be coupled to the reduced pressure inlet 770 and similarly coupled to the negative-pressure source 105 to fluidly couple the canister 115 to the negative-pressure source 105. The reduced pressure inlet 770 may be fluidly coupled to a channel 772 disposed on the first surface 716A of the second outer section 706A. The channel 772 may extend from the reduced pressure inlet 770 towards the first end 722A and towards the second end 724A. The channel 772 may be fluidly coupled to at least one chamber. In some embodiments, the channel 772 may be fluidly coupled to a first chamber 774 and a second chamber 776. The first chamber 774 may be located proximate to the first end 722A. In some embodiments, the second chamber 776 may be proximate to the second end 724A and opposite the first end 722A. The first chamber 774 may extend through the canister 115 from the second canister section 704 to the first canister section 702. More specifically, the first chamber 774 may extend through an opening 756A in a second portion 744A of the second filter carrier 708A and the opening 756 in the second portion 744 of the first filter carrier 708. The second chamber 776 may be disposed in the second canister section 704 and may extend from the second outer section 706A towards the second filter carrier 708A.

The first chamber 774 may have an opening disposed on an end 778 of the first chamber 774 opposite the second outer section 706A. The second chamber 776 may have an opening disposed on an end 780 of the second chamber 776 opposite the second outer section 706A. The opening on end 778 may be covered by a hydrophobic filter 782 and the opening on end 780 may be covered by a hydrophobic filter 784. The hydrophobic filter 782 may prevent ingress of liquids from the canister 115 into the first chamber 774 and the hydrophobic filter 784 may prevent ingress of liquids from the canister 115 into the second chamber 776. The volume of the first chamber 774 may be greater than the volume of the second chamber 776. In some embodiments, the plane associated with the end 778 of the first chamber 774 may be substantially parallel to the plane associated with the end 780 of the second chamber 776. The plane associated with the end 778 and the plane associated with the end 780 may be substantially parallel with the base 714A of the second outer section 706A. In other embodiments, the end 778 of the first chamber 774 and the end 780 of the second chamber 776 may be oriented such that the plane associated with the end 778 of the first chamber 774 may not be parallel to the plane associated with the end 780 of the second chamber 776. The first chamber 774 and second chamber 776 allow the passage of reduced pressure between the negative-pressure source 105 and the canister 115. In other embodiments, the canister 115 may not have the channel 772, the first chamber 774, or the second chamber 776. The reduced pressure inlet 770 may be covered by a hydrophobic filter on the first surface 716A of the second outer section 706A of the canister 115.

The second outer section 706A may further comprise a pathway connection or a therapy unit connection 786 disposed in the second outer section 706A. The therapy unit connection 786 may be configured to fluidly couple the canister 115 to the therapy unit 135. The therapy unit connection 786 may be configured to allow a fluid flow from the therapy unit 135 to reach the fluid flow pathway disposed between the first canister section 702 and the second canister section 704. The therapy unit connection 786 may be substantially circular. In other embodiments, the therapy unit connection 786 may be rectangular, ovular, triangular, or another shape. The therapy unit connection 786 may be substantially centered with respect to the width of the base 714A of the second outer section 706A but may be closer to the first end 722A than the second end 724A of the base 714A of the second outer section 706A. In other embodiments, the therapy unit connection 786 may be at a different location of the base 714A but may still fluidly couple the second outer section 706A to the therapy unit 135.

The second filter carrier 708A may comprise a first surface 736A and a second surface opposite the first surface 736A. The second filter carrier 708A may have a first end 738A and a second end 740A opposite the first end 738A. The first end 738A may have a width substantially equal to the width 720 of the first outer section 706 and the second end 740A may have a width substantially equal to the width 726 of the first outer section 706. In other embodiments, the first end 738A may have a width less than the width 720 and the second end 740A may have a width less than the width 726. The second filter carrier 708A may have a length that is substantially equal to the length 718 of the first outer section 706.

The first surface 736A of the second filter carrier 708A may be facing the second nonwoven layer 710A and the second surface of the second filter carrier 708A may be facing the second outer section 706A. The second filter carrier 708A may comprise a first portion 742A and a second portion 744A. The first portion 742A may extend from the second end 740A to the second portion 744A. The second portion 744A may extend from the first portion 742A to the first end 738A.

In some embodiments, a receiver or a notch 746A can be formed in the first end 738A. In some embodiments, the notch 746A extends through the second filter carrier 708A from the first surface 736A to the second surface. The notch 746A may be positioned proximate to a center of the width of the first end 738A. In other embodiments, the notch 746A may not be centered on the width of the first end 738A. In some embodiments, the notch 746A may have a width less than the width of the first end 738A. In some embodiments, the notch 746A may be aligned with the notch 732A of the second outer section 706A.

The first portion 742A of the second filter carrier 708A may have a first opening 748A and a second opening 788. There may be a periphery or a peripheral portion, such as a second filter boundary 750A surrounding and defining the first opening 748A. For example, the first opening 748A may comprise greater than 50% of the surface area of the first portion 742A. In some embodiments, the first opening 748A may comprise about 50% to 70% of the surface area of the first portion 742A. In other embodiments, the first opening 748A may be about 90% or greater of the surface area of the first portion 742A. A first support framework, such as a second filter section 752A can be disposed in the first opening 748A.

The second filter section 752A can comprise a plurality of arms, beams, or braces extending across the first opening 748A. In some embodiments, the second filter section 752A forms a plurality of holes 754A. Each hole 754A of the plurality of holes 754A may have a hexagonal shape. In some embodiments, each vertex of each hole 754A may be proximate to at least one vertex of an adjacent hole 754A. In other embodiments, the plurality of holes 754A may comprise different sizes and shapes. The plurality of holes 754A may maintain all or substantially all of the first opening 748A of the first portion 742A. The plurality of holes 754A of the second filter section 752A may be configured to allow fluid flow across the second filter section 752A.

The second opening 788 of the first portion 742A may be disposed between the first opening 748A and the second portion 744A of the second filter carrier 708A. The second opening 788 may be substantially centered along the width of the second filter carrier 708A. The second opening 788 may be configured to extend through the second filter carrier 708A and couple to the therapy unit connection 786 of the second outer section 706A. The second opening 788 may provide a sealed chamber between the second filter carrier 708A and the second outer section 706A for a fluid flow from the therapy unit 135 to reach the fluid flow pathway disposed between the first canister section 702 and the second canister section 704.

An opening 756A can be disposed in the second portion 744A. The opening 756A can be configured to provide fluid communication across the second filter carrier 708A through the second portion 744A. In some embodiments, the opening 756A can comprise a substantial portion of the second portion 744A. For example, the opening 756A may comprise greater than 50% of the surface area of the second portion 744A. In other embodiments, the opening 756A may be about 90% or greater of the surface area of the second portion 744A.

The second surface of the second filter carrier 708A may be coupled to the first surface 716A of the second outer section 706A. In other embodiments, the second filter carrier 708A may comprise a different size or shape but may still couple to the second outer section 706A to form the structure of the second canister section 704. In some embodiments, the second filter carrier 708A may be coupled to the second outer section 706A at one or more attachment points. In other embodiments, the second filter carrier 708A and the second outer section 706A may be coupled by compression gaskets, double sided adhesives, a weld, or any other suitable method of coupling to seal the second filter carrier 708A to the second outer section 706A.

The second nonwoven layer 710A may have a first surface 758A and a second surface opposite the first surface 758A. The second nonwoven layer 710A may be substantially the same shape as the first opening 748A of the first portion 742A of the second filter carrier 708A. A periphery 760A of the second nonwoven layer 710A may align with the second filter boundary 750A of the second filter carrier 708A. The second surface of the second nonwoven layer 710A may be configured to cover the first opening 748A of the first portion 742A of the second filter carrier 708A. The second surface of the second nonwoven layer 710A may be coupled to the first surface 736A of the second filter carrier 708A along the second filter boundary 750A.

The second evaporative section 712A may comprise one or more evaporative membrane layers such as a first evaporative layer 762A and a second evaporative layer 764A. The first evaporative layer 762A may comprise a first surface 766A and a second surface opposite the first surface 766A. The second evaporative layer 764A may have a first surface 768A and a second surface opposite the first surface 768A. The first evaporative layer 762A and the second evaporative layer 764A may be substantially the same size and shape as the second nonwoven layer 710A. The second surface of the first evaporative layer 762A may be configured to couple to the first surface 758A of the second nonwoven layer 710A. The second surface of the second evaporative layer 764A may be configured to couple to the first surface of the first evaporative layer 762A. In some embodiments, the second nonwoven layer 710A, the first evaporative layer 762A, and the second evaporative layer 764A may be welded to the second filter carrier 708A along the second filter boundary 750A. In other embodiments, the second nonwoven layer 710A, the first evaporative layer 762A, the second evaporative layer 764A, and the second filter carrier 708A may be coupled by other methods such as adhesives, compression gaskets, or other attachment methods.

The first outer section 706, the first filter carrier 708, the second filter carrier 708A, and the second outer section 706A may comprise a type of material having sufficient rigidity and structural integrity to withstand the reduced pressure required for negative-pressure treatment and to contain fluid therein. Some exemplary materials of the first outer section 706, the first filter carrier 708, the second filter carrier 708A, and the second outer section 706A are plastics, polymers, thermoplastics, metals, metal alloys, composition material, fiber-type materials, and other similar materials. The plastics described herein may be a substance or structure capable of being shaped or molded with or without the application of heat, a high polymer, usually synthetic, combined with other ingredients such as curatives, fillers, reinforcing agents, plasticizers, etc. Plastics can be formed or molded under heat and pressure in its raw state and machined to high dimensional accuracy, trimmed and finished in its hardened state. The thermoplastic type can be resoftened to its original condition by heat. In addition, the plastics may mean engineered plastics such as those that are capable of sustaining high levels of stress and are machinable and dimensionally stable. Some exemplary plastics are nylon, acetyls, polycarbonates, ABS resins, PPO/styrene, ISOPLAST 2530, TURLUX HS 2822, and polybutylene terephthalate. The thermoplastics described herein may be high polymers that soften when exposed to heat and return to their original condition when cooled to room temperature.

The first nonwoven layer 710 and the second nonwoven layer 710A may be comprised of a material of grade BK095620-11 having a weight of about 158 gsm. In other embodiments, the first nonwoven layer 710 and the second nonwoven layer 710A may comprise Libeltex TDL2 or a similar material that may allow fluids from the canister 115 to reach the first evaporative section 712 and the second evaporative section 712A. In some embodiments, the first nonwoven layer 710 and the second nonwoven layer 710A may each contain coatings or additives that may help to distribute fluid and increase fluid contact with the first evaporative section 712 and the second evaporative section 712A. The coatings may be polar so as to attract water molecules. The coatings may include nitrogen, oxygen, or fluorine to enable hydrogen bonding which may quickly remove the hydrogen molecules from the canister 115 and distribute them to the first evaporative section 712 and the second evaporative section 712A and into the airflow pathway. In other embodiments, the coatings may include halogens such as chlorine and bromine. Other coatings may include metal compounds or polymers such as sodium, potassium, or calcium. In some embodiments, the coatings may be plasma coatings or may be a corona treatment designed to oxidize the first nonwoven layer 710 and the second nonwoven layer 710A to provide a polar coating. The coatings may be applied to the first surface 758 of the first nonwoven layer 710 and to the second surface of the second nonwoven layer 710A. In other embodiments, the coatings may be applied to the first surface 758 and the second surface of the first nonwoven layer 710 and to the first surface 758A and the second surface of the second nonwoven layer 710A.

The first evaporative layer 762, the second evaporative layer 764, the first evaporative layer 762A, and the second evaporative layer 764A may be comprised of a high moisture vapor transmission rate (MVTR) polyurethane film. In some embodiments, the first evaporative layer 762, the second evaporative layer 764, the first evaporative layer 762A, and the second evaporative layer 764A may be comprised of film such that the first evaporative section 712 and the second evaporative section 712A are about 70 μm thick. In other embodiments, the first evaporative section 712 and the second evaporative section 712A may each comprise only one layer of film that is about 40-50 μm thick. In some embodiments, the high MVTR film may be BASF E2385A 72000. In some embodiments, the high MVTR film may optimally have an MVTR of about 7000 to 5000 g/m²/24hrs. In other embodiments, the high MVTR film may be COVESTRO VPT 9121. The thickness of the COVESTRO VPT 9121 film may be about 15 to 100 μm. In other embodiments, the high MVTR film may be COVESTRO Platilon U 250 μm.

The canister 115 may be formed by coupling the first canister section 702 to the second canister section 704. When the first canister section 702 is coupled to the second canister section 704, the airflow pathway may be created between the first canister section 702 and the second canister section 704. The airflow pathway may be fluidly connected to the therapy unit 135 through the chamber created between the second opening 788 of the first portion 742A of the second filter carrier 708A and the therapy unit connection 786 of the second outer section 706A. The airflow pathway may extend from the second opening 788 of the first portion 742A of the second filter carrier 708A to an end of the canister 115. The airflow pathway may be about 2 mm thick. The airflow pathway may extend through the canister 115 and open to ambient environment at the end of the canister 115 to allow evaporated fluids from the canister 115 to escape to the ambient environment. The first evaporative section 712 and the second evaporative section 712A may line the airflow pathway. The more liquid from the canister 115 that can reach the first evaporative section 712 and the second evaporative section 712A, the greater the amount of fluid that can be evaporated from the canister 115.

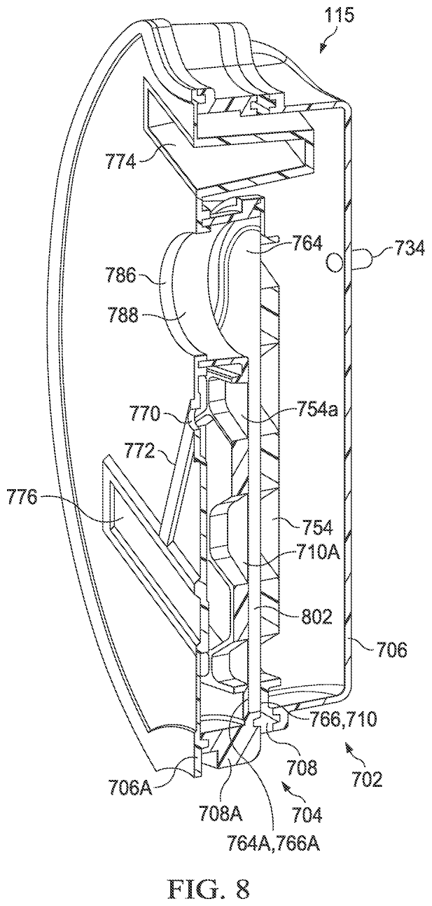

FIG. 8 is a sectional perspective view of the canister 115, illustrating additional details that may be associated with some embodiments of the therapy system 100. The exterior structure of the canister 115 is comprised of the first outer section 706, the first filter carrier 708, the second filter carrier 708A, and the second outer section 706A. The airflow pathway 802 is disposed between the first portion 742 of the first filter carrier 708 and the first portion 742A of the second filter carrier 708A. The airflow pathway 802 may be lined by the first evaporative section 712 and the second evaporative section 712A. The therapy unit connection 786 may be configured to connect to the fluid outlet 222 of the second wall 212 of the therapy unit 135.

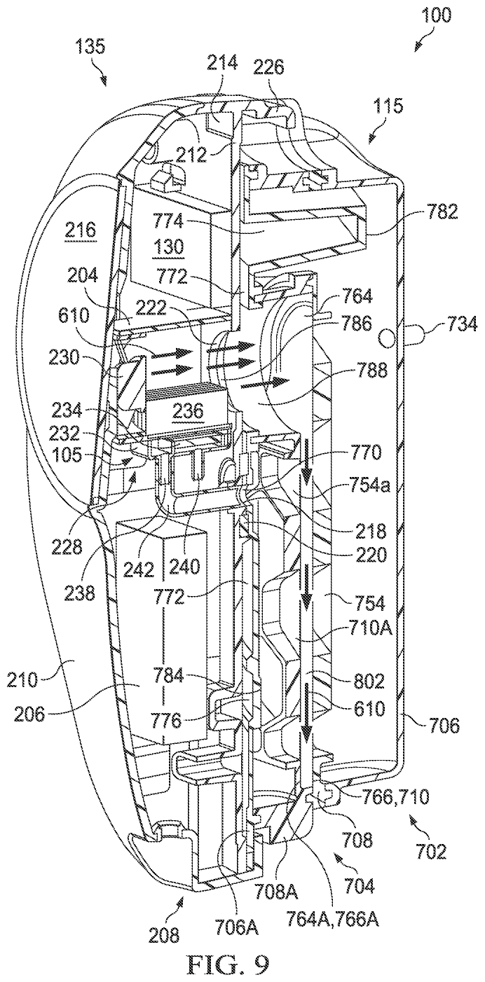

FIG. 9 illustrates an operative embodiment of the therapy unit 135 and the canister 115. In some embodiments, the canister 115 may process a volume of liquids received from a tissue site that is greater than a volume of the canister 115. The canister 115 may include features that enable evaporation of liquids received inside the canister 115. The canister 115 can be coupled to the therapy unit 135 at the attachment points 226. The controller 130 may operate the pump 234 of the pump module 228 to draw fluid from the tissue site through the dressing 110. The fluid may flow from the dressing 110 through a conduit configured to be fluidly connected to the dressing 110 and the fluid inlet 734. The fluid may flow through the conduit and into the canister 115 through the fluid inlet 734. Liquids from the fluid may collect in the canister 115 while gasses from the fluid may flow through the hydrophobic filter 782 of the first chamber 774 and the hydrophobic filter 784 of the second chamber 776 of the canister 115. Gasses from the first chamber 774 may travel through the channel 772 to the reduced pressure inlet 770 and gasses from the second chamber 776 may travel through the channel 772 to the reduced pressure inlet 770. The gasses may then flow through the reduced pressure inlet 770 to the intake conduit 242 of the therapy unit 135.

Upon reaching the intake conduit 242, the gasses may flow through the intake conduit 242 from the second wall 212 of the therapy unit 135 to the intake 238 of the pump module 228. The gasses may enter the pump module 228 from the intake 238 and may exit the pump module 228 through the exhaust 240. The gasses may exit the exhaust 240 into the interior space 214 of the therapy unit 135. In some embodiments, the gasses may then escape to the ambient air surrounding the therapy system 100 from the interior space 214 of the therapy unit 135 through a vent located in the first wall 210 or the second wall 212. In other embodiments, there may be a fluid conductor that may fluidly couple the exhaust 240 to the fluid outlet 222. In these embodiments, the gasses may flow from the exhaust 240 through the fluid conductor to the fluid outlet 222. From the fluid outlet 222, the gasses may flow through the therapy unit connection 786 to the chamber created between the therapy unit connection 786 and the second opening 788 of the first portion 742A of the second filter carrier 708A. The gasses may then flow through the second opening 788 of the first portion 742A of the second filter carrier 708A and into the airflow pathway 802. From the airflow pathway 802, the gasses may exit to the ambient environment surrounding the therapy system 100.

The pump 234 may generate heat as the pump 234 draws fluid from the tissue site. The controller 130 may receive a signal from the temperature sensor 302 indicative of a temperature of the pump module 228. If the signal from the temperature sensor 302 reaches a threshold temperature, the controller 130 may operate the forced-air device 230 to generate a fluid flow through the pathway enclosure 204, for example, the fluid flow 610. The fluid flow 610 may flow through the pathway enclosure 204 to the fluid outlet 222 of the second wall 212 of the therapy unit. The fluid flow 610 may then flow through the therapy unit connection 786 of the second outer section 706A of the canister 115. The fluid flow 610 may then flow through the second opening 788 of the first portion 742A of the second filter carrier 708A and into the airflow pathway 802. The fluid flow 610 may further be directed through the airflow pathway 802 to ambient air surrounding the canister 115. The exhaust 240 of the pump module 228 may be configured to release any exhaust from the pump module 228 into the therapy unit 135. As the fluid flow 610 is directed through the pathway enclosure 204, the fluid flow 610 is warmed as it crosses the lid 236 and flows through the plurality of ribs 326. As the fluid flow 610 reaches the first evaporative section 712 and the second evaporative section 712A, convection heating of the first evaporative section 712 and the second evaporative section 712A and the movement of the fluid may cause moisture vapor passing by osmosis through the first evaporative section 712 and the second evaporative section 712A to transmit into the fluid flow 610. The fluid flow 610 may carry away the moisture vapor, allowing more liquid within the canister 115 to evaporate and pass through the first evaporative section 712 and the second evaporative section 712A.

In other embodiments, additional elements within the therapy unit 135 may generate heat. For example, the controller 130, the power source 206, the pump module 228, the pathway enclosure 204, the forced-air device 230, and the pump 234 may generate heat. Airflow through the therapy unit 135 can be directed across the controller 130 or the power source 206, for example, so that the heat generated by these components may be transferred into the fluid flow 610. The additional heat may be directed through the pathway enclosure 204 and into the canister 115. In some embodiments, heat sinks similar to the lid 236 may be used to transfer the generated heat into the fluid flow 610 and used to encourage evaporation of liquids received inside the canister 115.

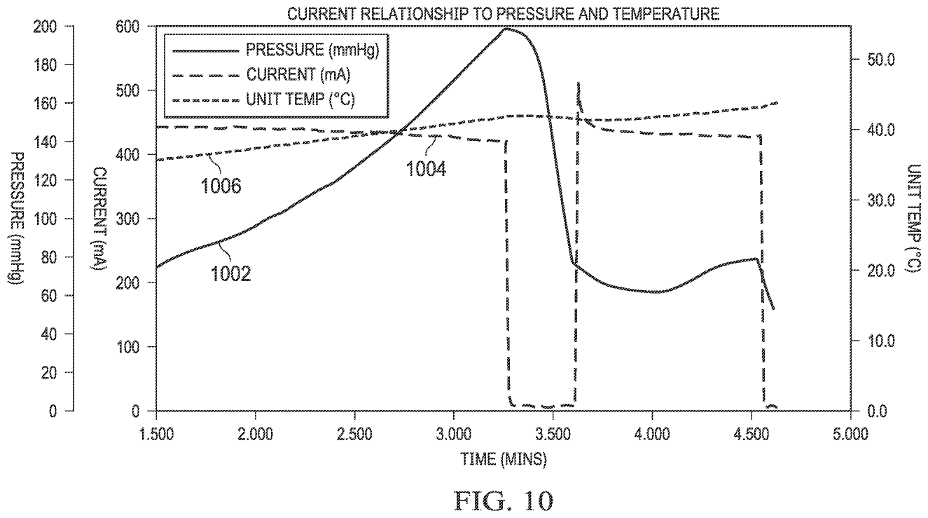

FIG. 10 is a graphical representation illustrating the relationship between pump pressure, electrical energy consumed, and temperature with respect to time. In the graph of FIG. 10, the negative pressure in millimeters of mercury (mmHg) generated by the pump module 228, the required current in milliamperes (mA), and the corresponding temperature in degrees Celsius° C. are represented on the Y-axis. The X-axis represents the elapsed time of operation in minutes. The negative-pressure of the pump module 228 over time is represented by line 1002. The electrical energy consumed by the pump module 228 is represented by line 1004, and the temperature of the pump module 228 is represented by line 1006. As illustrated in FIG. 10, as the temperature increased during operation of the pump module 228, the electrical energy required to produce the desired negative pressure decreased. Thus, as the temperature of the pump module 228 increased, the pump module 228 became more efficient.

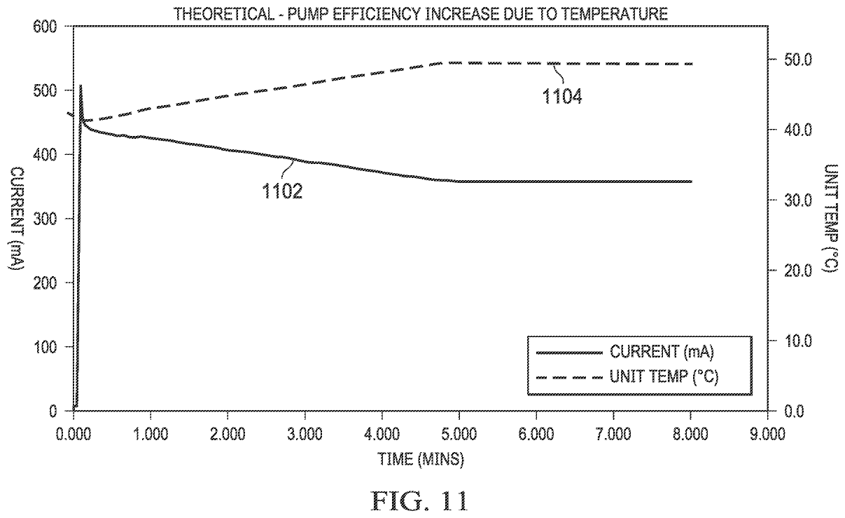

FIG. 11 is a graphical representation illustrating the relationship between electrical energy consumed and temperature with respect to time. In the graph of FIG. 11, the required current in milliamperes (mA) and the corresponding temperature in degrees Celsius° C. are represented on the Y-axis. The X-axis represents the elapsed time of operation in minutes. The electrical energy consumed by the pump module 228 is represented by line 1102, and the temperature of the pump module 228 is represented by line 1104. As illustrated in FIG. 11, as the temperature increased during operation of the pump module 228, the electrical energy required over time decreased as the temperature increased. The relationship may be extrapolated up to a theoretical limit after which the increase in temperature would negatively impact the pump module 228. In the illustrated embodiments, the controller 130 can operate the forced-air device 230 to maintain the temperature of the pump module below the temperature of decreasing returns of pump efficiency.

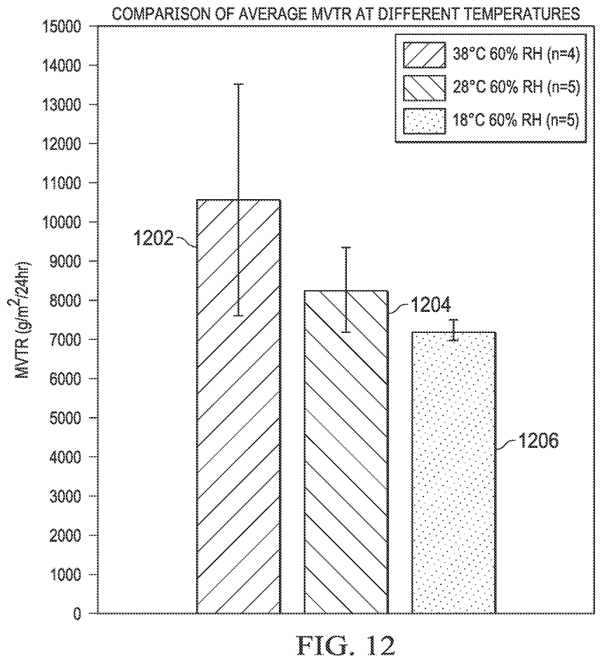

FIG. 12 is a graphical representation of the moisture vapor transmission rate (MVTR) of the canister 115 for various temperatures. As shown in FIG. 12, an increase in the temperature of the fluid flow 610 flowing through the airflow pathway 802 leads to an increase in the MVTR of the canister 115. As shown by bar 1202, where the temperature was about 38° C. at 60% relative humidity, the MVTR of the canister 115 was the greatest, the decreased temperatures of bar 1204 at about 28° C. and bar 1206 at 18° C. had lower MVTR. Providing fluid flow from the therapy unit 135 through the canister 115 and scavenging heat from the operation of the pump module 228 through the plurality of ribs 326 can increase evaporation of fluids from the canister 115, permitting the canister 115 to process more fluids than the volume of the canister 115 can hold at any one time.

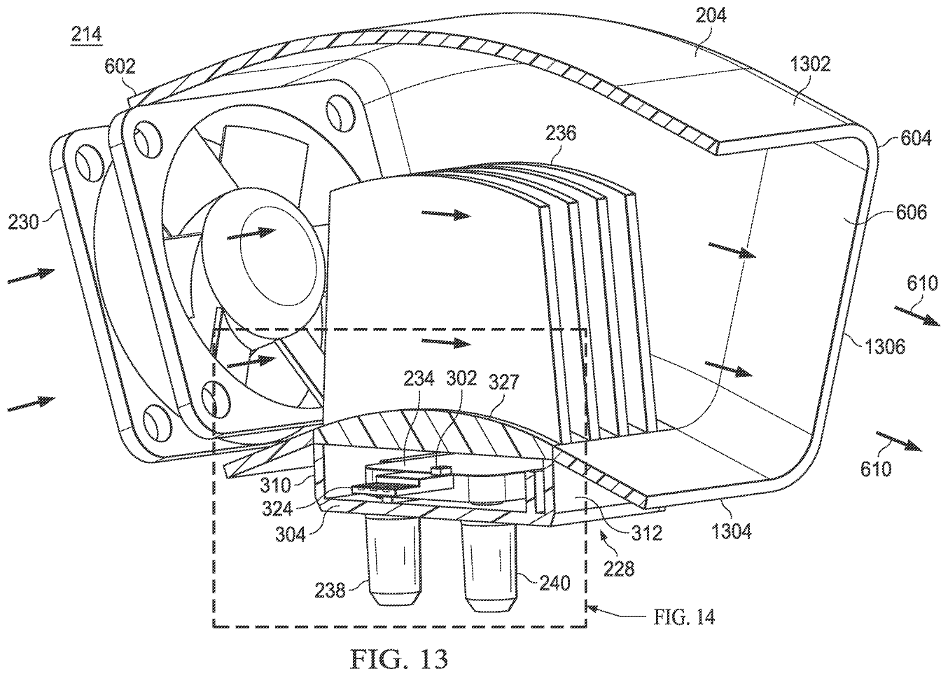

FIG. 13 is a perspective cutaway view of a portion of the therapy unit 135 illustrating additional details that may be associated with some embodiments. The first end 602 of the pathway enclosure 204 may be adjacent to the first wall 210, and the second end 604 of the pathway enclosure 204 may be disposed adjacent to the fluid outlet 222 of the second wall 212. The pathway enclosure 204 may have a first wall 1302 that has a generally convex shaped exterior facing away from a second wall 1304 and a generally concave shaped interior facing the second wall 1304. The second wall 1304 may have a generally convex shaped interior facing the first wall 1302 and a generally concave shaped exterior facing away from the first wall 1302. A third wall 1306 may extend from the first wall 1302 to the second wall 1304. A fourth wall not visible in this view may be opposite the third wall 1306 and extend from the first wall 1302 to the second wall 1304. The volume 606 of the pathway enclosure 204 may be defined by the first wall 1302, the second wall 1304, the third wall 1306, and the fourth wall.

The forced-air device 230 can be positioned in the volume 606 adjacent to the first end 602. The forced-air device 230 may be coupled to the interior side of the second wall 1304 and thus may be sitting at a non-perpendicular angle with respect to the pump module 228. The forced-air device 230 can be an axial fan, a centrifugal fan, a cross-flow fan, a bellows, a coanda effect device, a convective airflow device, or an electrostatic airflow device, or other similar device configured to generate a fluid flow. The pathway enclosure 204 may surround the forced-air device 230, generally directing the fluid flow generated by the forced-air device 230 from the first end 602 to the second end 604 of the pathway enclosure 204 while maintaining the fluid flow within the volume 606.

An opening 608 can be disposed in the second wall 1304 of the pathway enclosure 204. The opening 608 can be sized so that the lid 236 can be inserted into the volume 606 of the pathway enclosure 204. The second surface 327 of the lid 236 may be flush with the interior of the second wall 1304 of the pathway enclosure 204. The second surface 327 of the lid 236 may have a generally convex shape facing the first wall 1302. The convex shape of the second surface 327 of the lid 236 may have the same curve as the convex shape of the interior of the second wall 1304. The plurality of ribs 326 can project from the second surface 327 of the lid 236 into the volume 606 of the pathway enclosure 204. In some embodiments, the plurality of ribs 326 may be curved in a generally convex shape at an end of the plurality of ribs 326 opposite the second wall 1304 of the pathway enclosure 204. The curve of the plurality of ribs 326 may be substantially the same as the curve of the second surface 327 of the lid 236 and the interior of the second wall 1304. In some embodiments, the radius of curvature of the second wall 1304, the second surface 327 of the lid 236, and the plurality of ribs 326 may be between about 10 mm and about 50 mm. In other embodiments, the radius of curvature of the second wall 1304, the second surface 327 of the lid 236, and the plurality of ribs 326 may be larger than 50 mm. In other embodiments, the end of the plurality of ribs opposite the second wall 1304 of the pathway enclosure 204 may not be curved and may be substantially the same as described in FIGS. 3-5.

The forced-air device 230 can be oriented relative to the lid 236 to permit the forced-air device 230 to generate a fluid flow 610 through the plurality of ribs 326. The fluid flow 610 may follow the curve of the first wall 1302 and the second wall 1304 of the pathway enclosure 204. This fluid flow 610 may be more concentrated in the area of the plurality of ribs 326 closer to the second wall 1304 of the pathway enclosure 204 than the area of the plurality of ribs 326 closer to the first wall 1302 of the pathway enclosure. In other embodiments, not pictured herein, a portion of the first wall 1302 proximate to the end of the plurality of ribs 326 opposite the second wall 1304 may be substantially perpendicular to the base 304 of the pump module 228. This configuration of the pathway enclosure 204 may force the fluid flow 610 through the plurality of ribs 326, resulting in an increased temperature of the fluid flow 610. In other embodiments, the pathway enclosure 204 may have other configurations designed to force the fluid flow 610 through the plurality of ribs 326 of the lid.

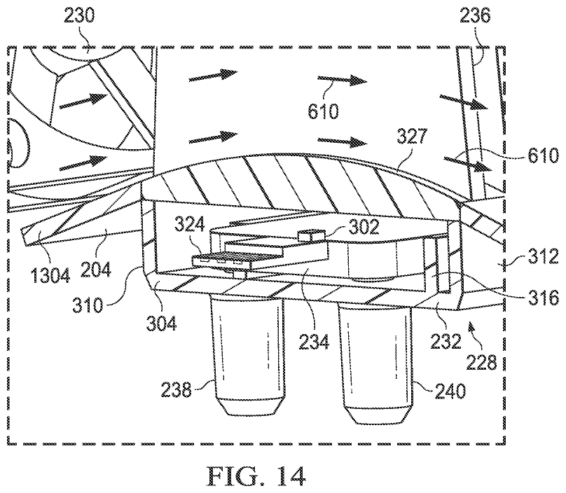

FIG. 14 is a perspective cut-away view of a portion of the pathway enclosure 204, the forced-air device 230, and the pump module 228 of FIG. 13. The fluid flow 610 is more concentrated in the area of the plurality of ribs 326 closer to the second wall 1304 of the pathway enclosure 204 than the area of the plurality of ribs 326 closer to the first wall 1302 of the pathway enclosure 204. This curved fluid flow 610 may be more efficient because it uses principles of air entrainment. For example, fluid proximate to the ribs 326 may have a higher temperature than fluid within the fluid flow 610. The curvature of the lid 236 and the pathway enclosure 204 can cause turbulence within the fluid flow 610, leading to an increase of mixing between the higher temperature fluid adjacent the ribs 326 and the remainder of the fluid flow 610. This orientation may lead to a more efficient cooling of the lid 236 of the pump module 228 which may reduce the energy that is required to cool the lid 236 of the pump module 228. This orientation may also lead to a warmer fluid flow 610 being directed away from the lid 236 and into the airflow pathway 802 of the canister 115.

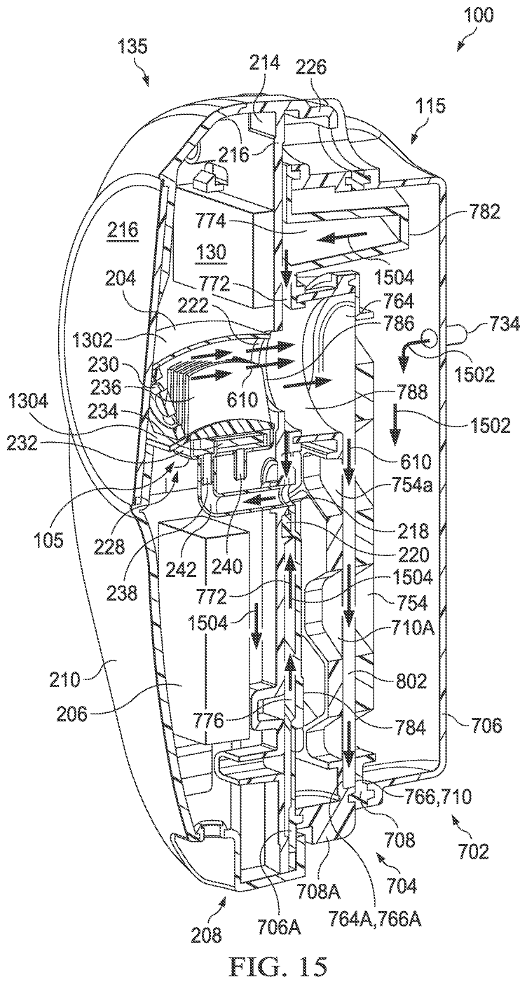

FIG. 15 illustrates an operative embodiment of the therapy unit 135 and the canister 115. In some embodiments, the canister 115 may process a volume of liquids received from a tissue site that is greater than a volume of the canister 115. The canister 115 may include features that enable evaporation of liquids received inside the canister 115. The canister 115 can be coupled to the therapy unit 135 at the attachment points 226. The controller 130 may operate the pump 234 of the pump module 228 to draw fluid 1502 from the tissue site through the dressing 110. The fluid 1502 may flow from the dressing through a conduit configured to be fluidly connected to the dressing and the fluid inlet 734. The fluid 1502 may flow through the conduit and into the canister 115 through the fluid inlet 734. Liquids from the fluid 1502 may collect in the canister 115 while gasses 1504 from the fluid 1502 may flow through the hydrophobic filter 782 of the first chamber 774 and the hydrophobic filter 784 of the second chamber 776 of the canister 115. Gasses 1504 from the first chamber 774 may travel through the channel 772 to the reduced pressure inlet 770 and gasses 1504 from the second chamber 776 may travel through the channel 772 to the reduced pressure inlet 770. The gasses 1504 may then flow through the reduced pressure inlet 770 to the intake conduit 242 of the therapy unit 135.

Once the gases reach the intake conduit 242 of the therapy unit 135, the gasses 1504 may flow through the intake conduit 242 from the second wall 212 of the therapy unit 135 to the intake 238 of the pump module 228. The gasses 1504 may enter the pump module 228 from the intake 238 and may exit the pump module 228 through the exhaust 240. The gasses 1504 may exit the exhaust 240 into the interior space 214 of the therapy unit 135. In some embodiments, the gasses 1504 may then escape to the ambient air surrounding the therapy system 100 from the interior space 214 of the therapy unit 135 through a vent located in the first wall 210 or the second wall 212. In other embodiments, there may be a fluid conductor that may fluidly couple the exhaust 240 to the fluid outlet 222. In these embodiments, the gasses 1504 may flow from the exhaust 240 through the fluid conductor to the fluid outlet 222. From the fluid outlet 222, the gasses 1504 may flow through the therapy unit connection 786 to the chamber created between the therapy unit connection 786 and the second opening 788 of the first portion 742A of the second filter carrier 708A. The gasses 1504 may then flow through the second opening 788 of the first portion 742A of the second filter carrier 708A and into the airflow pathway 802. From the airflow pathway 802, the gasses 1504 may exit to the ambient environment surrounding the therapy system 100.

The pump 234 may generate heat as the pump 234 draws fluid from the tissue site. The controller 130 may receive a signal from the temperature sensor 302 indicative of a temperature of the pump module 228. If the signal from the temperature sensor 302 reaches a threshold temperature, the controller 130 may operate the forced-air device 230 to generate a fluid flow through the pathway enclosure 204, for example, the fluid flow 610. The fluid flow 610 may flow through the pathway enclosure 204. The fluid flow 610 may be more concentrated in the area of the plurality of ribs 326 closer to the second wall 1304 of the pathway enclosure 204 than the area of the plurality of ribs 326 closer to the first wall 1302 of the pathway enclosure 204 because the pathway enclosure 204 is curved. The fluid flow 610 may flow through the pathway enclosure 204 to the fluid outlet 222 of the second wall 212 of the therapy unit. The fluid flow 610 may then flow through the therapy unit connection 786 of the second outer section 706A of the canister 115. The fluid flow 610 may then flow through the second opening 788 of the first portion 742A of the second filter carrier 708A and into the airflow pathway 802. The fluid flow 610 may further be directed through the airflow pathway 802 to ambient air surrounding the canister 115. The exhaust 240 of the pump module 228 may be configured to release any exhaust from the pump module 228 into the therapy unit 135. As the fluid flow 610 is directed through the pathway enclosure 204, the fluid flow 610 is warmed as it crosses the lid 236 and flows through the plurality of ribs 326. As the fluid flow 610 reaches the first evaporative section 712 and the second evaporative section 712A, convection heating of the first evaporative section 712 and the second evaporative section 712A and the movement of the fluid may cause moisture vapor passing by osmosis through the first evaporative section 712 and the second evaporative section 712A to transmit into the fluid flow 610. The fluid flow 610 may carry away the moisture vapor, allowing more liquid within the canister 115 to evaporate and pass through the first evaporative section 712 and the second evaporative section 712A.

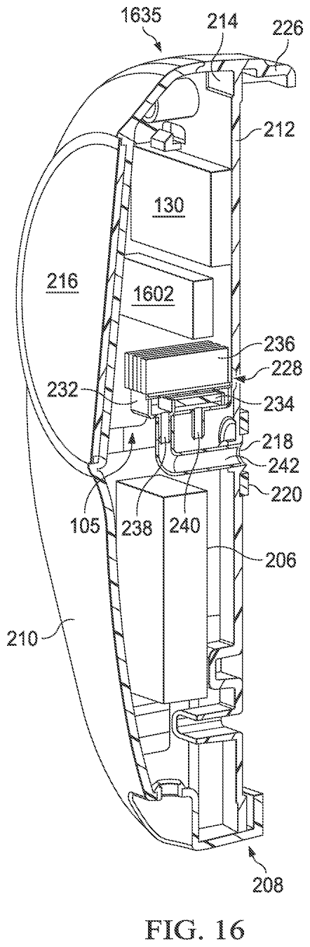

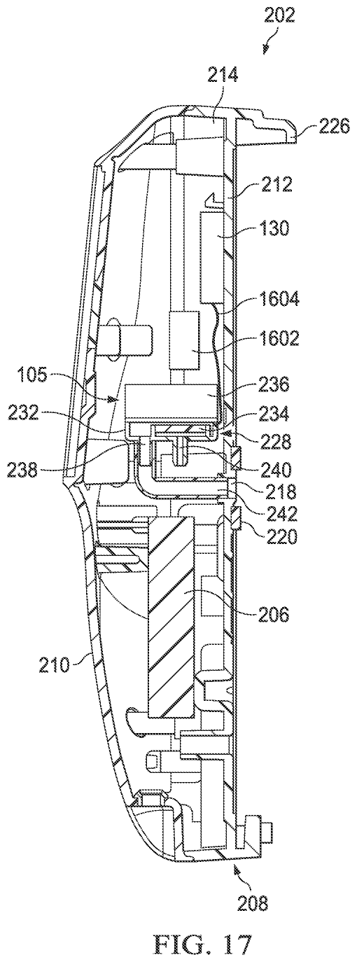

FIGS. 16 and 17 are sectional views of another therapy unit 1635 illustrating additional details that may be associated with some embodiments. The therapy unit 1635 may be similar to and operated as described with respect to the therapy unit 135. As shown in FIGS. 16 and 17, the therapy unit 1635 does not include the forced-air device 230 and the fluid outlet 222. The therapy unit 1635 can include the pump module 228, the power source 206, the controller 130, and a diaphragm pump 1602. The power source 206 and the controller can be coupled, for example, by electric coupling or communicative coupling to the pump module 228 and the diaphragm pump 1602. In some embodiments, the controller 130 and the pump module 228 may be coupled by a ribbon cable such as ribbon cable 1604.

The diaphragm pump 1602 may operate in conjunction with the pump 234. The diaphragm pump 1602 may be fluidly connected to the intake conduit 242 in order to operate in conjunction with the pump 234. If the therapy unit 1635 is operated with both the pump 234 and the diaphragm pump 1602, the dressing 110 may be drawn to a desired negative-pressure faster than if either the pump 234 or the diaphragm pump 1602 were operated on their own. Including the diaphragm pump 1602 may increase the noise output of the therapy unit 135. Despite the increased noise output of the therapy unit 1635, including the diaphragm pump 1602 may be desirable because including the diaphragm pump 1602 may increase the lifespan of the pump 234 by reducing the strain on the pump 234.

The systems, apparatuses, and methods described herein may provide significant advantages. For example, using piezoelectric pumps in therapy units 135 may result in quieter, smaller, and lighter therapy units 135 that may be more desirable to patients. Additionally, placing the pump 234 into the pump module 228 may make the pump 234 significantly more efficient by maintaining an optimal temperature inside the pump module 228 for the pump 234 to operate at. Further, the heat output of the pump 234 may be harnessed to increase evaporation of fluids stored in canister 115. The canister 115 may optimize the amount of fluid that can be drawn from a tissue site by directing the heat output from the pump 234 over the first evaporative section 712 and the second evaporative section 712A which may allow for dressing 110 to stay in place at a tissue site for longer periods of time which may increase patient comfort and decrease the environmental impact of using the therapy system 100. Overall, utilizing the heat output of the pump 234 as described in the embodiments herein may result in a quieter, lighter, smaller, and more cost effective therapy system 100.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the canister 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for negative-pressure therapy, the system comprising:
a dressing configured to be positioned adjacent to a tissue site;
a therapy unit comprising:
a pump module, the pump module comprising:
a pump casing:
a piezoelectric pump coupled to the pump casing; and
a lid coupled to the pump casing, the lid configured to seal the piezoelectric pump between the pump casing and the lid;
a forced air module, the forced air module comprising:
a forced air device positioned proximate to the pump module, the forced air device configured to generate a fluid flow; and
a pathway enclosure configured to form a fluid path across the lid; and
a canister, the canister comprising:
a pathway connection, the pathway connection configured to allow fluid communication between the pathway enclosure and the canister;
an airflow pathway extending through the canister from the pathway connection; and
an evaporative membrane lining the airflow pathway.

2. The system of claim 1, the pump casing comprising:
a base with a first side and a second side opposite the first side;
a first wall protruding from the first side of the base, a second wall opposite the first wall, a third wall perpendicular to and extending from the first wall to the second wall, and a fourth wall opposite the third wall;
a walled enclosure disposed inboard of the first wall, the second wall, the third wall, and the fourth wall defining a pump seat;
a first bore depending through the base from the pump seat on the first side of the base to the second side of the base;
a second bore depending through the base;
a first conduit extending from the second side of the base, the first conduit having at least one lumen fluidly coupled to the first bore; and
a second conduit extending from the second side of the base, the second conduit having at least one lumen fluidly coupled to the second bore.

3. The system of claim 2, wherein the piezoelectric pump is coupled to the first side of the base at the pump seat.

4. The system of claim 3, the pump module further comprising a temperature sensor, the temperature sensor disposed between the pump casing and the lid.

5. The system of claim 2, wherein the pump casing comprises an insulating material.

6. The system of claim 1, wherein the lid comprises a heat sink.

7. The system of claim 2, wherein the lid comprises a plurality of ribs extending away from the pump module opposite the pump casing.

8. The system of claim 7, wherein the plurality of ribs extend from the third wall to the fourth wall such that the ribs are parallel to the first wall and the second wall of the pump casing.

9. The system of claim 8, wherein the forced air device is configured to direct the fluid flow through the ribs the lid.

10. The system of claim 9, wherein the forced air device is positioned relative to the third wall of the pump casing at a non-perpendicular angle.

11. The system of claim 10, wherein the fluid flow defines a curved pathway.

12. The system of claim 1, wherein the forced air device is an axial fan.

13. The system of claim 1, the therapy unit further comprising a diaphragm pump proximal to the pump module.

14. The system of claim 13, wherein the diaphragm pump is configured to operate in conjunction with the piezoelectric pump.

15. The system of claim 1, wherein the pathway enclosure comprises a rigid plastic.

16. A pump module comprising:
a pump casing comprising:
a base with a first side and a second side opposite the first side;
a first wall protruding from the first side of the base, a second wall opposite the first wall, a third wall perpendicular to and extending from the first wall to the second wall, and a fourth wall opposite the third wall;
a walled enclosure disposed inboard of the first wall, the second wall, the third wall, and the fourth wall defining a pump seat;
a first bore depending through the base from the pump seat on the first side of the base to the second side of the base;
a second bore depending through the base;
a first conduit extending from the second side of the base, the first conduit having at least one lumen fluidly coupled to the first bore; and
a second conduit extending from the second side of the base, the second conduit having at least one lumen fluidly coupled to the second bore;
a piezoelectric pump coupled to the first side of the base at the pump seat;
a lid coupled to the pump casing, the lid configured to seal the piezoelectric pump between the pump casing and the lid; and
a temperature sensor disposed between the pump casing and the lid;
wherein the pump casing and the lid are configured to optimize an [[the]] efficiency of the piezoelectric pump.

17. The pump module of claim 16, wherein the pump casing further comprises an opening extending through the first wall.

18. The pump module of claim 17, further comprising:
a projection extending from the piezoelectric pump, the projection configured to extend through the opening in the first wall; and
an electrical connection coupled to a first side of the projection, the electrical connection configured to be coupled to a potential source outside of the pump casing.

19. The pump module of claim 16, wherein the temperature sensor is coupled to the piezoelectric pump opposite of the pump seat.

20.-43. (canceled)

44. A method for generating negative pressure comprising:
positioning a dressing adjacent to a tissue site;
coupling a therapy unit having a pump module to the dressing, the pump module comprising:
a pump casing, the pump casing comprising:
a base with a first side and a second side opposite the first side;
a first wall protruding from the first side of the base, a second wall opposite the first wall, a third wall perpendicular to and extending from the first wall to the second wall, and a fourth wall opposite the third wall;
a walled enclosure disposed inboard of the first wall, the second wall, the third wall, and the fourth wall defining a pump seat;
a first bore depending through the base from the pump seat on the first side of the base to the second side of the base;
a second bore depending through the base;
an exhaust conduit extending from the second side of the base, the exhaust conduit having at least one lumen fluidly coupled to the first bore; and
an intake conduit extending from the second side of the base, the intake conduit having at least one lumen fluidly coupled to the second bore;
a piezoelectric pump coupled to the first side of the base at the pump seat;
a lid coupled to the pump casing, the lid configured to seal the piezoelectric pump between the pump casing and the lid; and
a temperature sensor disposed between the pump casing and the lid;
drawing the tissue site to a desired negative pressure comprising:
starting the piezoelectric pump;
starting a diaphragm pump, the diaphragm pump positioned proximate to the pump module in the therapy unit and configured to be in fluid communication with the tissue site;
using the intake conduit to draw fluid from the tissue site into the pump module;
using the exhaust conduit to allow the fluid from the pump module to escape from the pump module;
operating a forced air device positioned proximate to the pump module to regulate an internal temperature of the pump module comprising:
monitoring the internal temperature of the pump module with the temperature sensor;
if the internal temperature is greater than a predetermined temperature, starting a forced air device;
using the forced air device to direct a fluid flow over the lid, the fluid flow configured to reduce the internal temperature of the pump module; and
if the internal temperature is less than the predetermined temperature, stopping the forced air device.

45.-46. (canceled)
